(19) United States
(12) Patent Application Publication
(10) Pub. No.: US 2023/0287226 A1
(43) Pub. Date: Sep. 14, 2023

(54) Electrochemical Biosensors for Rapid and Sensitive Detection of Pathogens and Pathogenic Biomarkers

(71) Applicants: Carnegie Mellon University, Pittsburgh, PA (US); University of Pittsburgh - Of the Commonwealth System of Higher Education, Pittsburgh, PA (US)

(72) Inventors: Rahul Panat, Sewickley, PA (US); Shou-Jiang Gao, Pittsburgh, PA (US); Azahar Ali, Pittsburgh, PA (US); Chunshan Hu, Pittsburgh, PA (US); Bin Yuan, Pittsburgh, PA (US); Mohammad Sadeq Saleh, Ithaca, NY (US)

(21) Appl. No.: 18/013,951

(22) PCT Filed: Jul. 2, 2021
(86) PCT No.: PCT/US2021/040302

§ 371 Date: Dec. 30, 2022

Related U.S. Application Data
(60) Provisional application No. 63/047,368, filed on Jul. 2, 2020.
(60) Provisional application No. 63/050,182, filed on Jul. 10, 2020.
(60) Provisional application No. 63/076,174, filed on Sep. 9, 2020.

(51) Int. Cl. C09D 11/04; G01N 27/327; H01B 5/14; G01N 33/543; G01N 33/569; C09D 11/52; C09D 11/30; B41M 5/00

(52) U.S. Cl. CPC C09D 11/04; G01N 27/3276; H01B 5/14; G01N 33/5438; G01N 33/56983; C09D 11/52; C09D 11/30; B41M 5/0082; G01N 2469/10

(57) ABSTRACT

A method of preparing a functionalized electrode array is provided. The method includes depositing a conductive material onto the surface of a substrate by droplet-based printing of particles comprising an electrically-conductive material. The surface of the conductive material is functionalized with a binding reagent that binds to an analyte. A three-dimensional electrode array and microfluidic test device are also provided.

### CROSS REFERENCE TO RELATED APPLICATIONS

This application is the United States national phase of International Application No. PCT/US2021/040302 filed Jul. 2, 2021, and claims priority to U.S. Provisional Application No. 63/047,368 filed Jul. 2, 2020, U.S. Provisional Application No. 63/050,182 filed Jul. 10, 2020, and U.S. Provisional Application No. 63/076,174 filed Sep. 9, 2020, the disclosures of which are hereby incorporated by reference in their entireties.

### BACKGROUND OF THE INVENTION

Pandemics and epidemics caused by emerging infectious agents such as SARS-CoV-2, influenza viruses, Zika virus, and Ebola virus have seriously affected human health, and led to lost economic activity and challenged health-care systems. Early and accurate detection of infectious diseases, such as COVID-19, can help contain pandemics and reduce fatality rates.

Two primary approaches have been used to detect a COVID-19 infection. The first approach utilizes the genetic sequence of the virus and includes genomic sequencing, CRISPR-based test, and reverse transcription real time quantitative PCR. These approaches are relatively accurate but are generally slow because of the need for multiple sample processing steps and are dependent upon the protocol of sampling, preservation, and transportation. The second approach is to detect specific antibodies to viral antigens through serological methods, such as enzyme-linked immunosorbent assay (ELISA) and lateral flow immunoassay. These assays are based on structural proteins including spike protein (S-protein) and/or nucleocapsid protein (N-protein) and have a relatively low sensitivity. These assays are also incapable of detecting an infectious disease in the early stage (2-5 days) of infection or for someone who takes longer to develop antibodies.

Superior methods of detecting infectious diseases within days of infection are desirable, especially those that allow for detection of infectious diseases within minutes or seconds.

### SUMMARY OF THE INVENTION

A method of preparing a functionalized electrode is provided. The method comprising: depositing a conductive material onto the surface of a substrate by droplet-based printing, such as aerosol jet printing, of particles comprising an electrically-conductive material, and functionalizing the surface of the conductive material with a binding reagent that binds to an analyte.

In another aspect or embodiment, an electrode for sensing an analyte is provided, comprising: a substrate comprising a droplet-based printed, and optionally sintered, conductive material, and a coating comprising a binding reagent covalently bonded to the surface of the conductive material that binds to an analyte.

In another aspect or embodiment, a microfluidic test device is provided, comprising: one or more sensing electrodes in a chamber or channel configured to receive a liquid test sample, wherein the sensing electrode comprises a substrate and an electrode array comprising a working electrode, a counter electrode, and, optionally, a reference electrode on the substrate, and the working electrode comprises a droplet-based printed, and optionally sintered conductive material, and a coating comprising a binding reagent covalently bonded to the surface of the conductive material that binds to an analyte.

In another aspect or embodiment, a method of sensing an analyte is provided, the method comprising: contacting a fluid comprising the analyte with the electrode comprising: a substrate comprising a droplet-based printed, and optionally sintered, conductive material, and a coating comprising a binding reagent covalently bonded to the surface of the conductive material that binds to an analyte.

Non-limiting aspects of the invention will now be described in the following numbered clauses:

Clause 1: A method of preparing a functionalized electrode, comprising:
depositing a conductive material onto the surface of a substrate by droplet-based printing, such as aerosol jet printing, of particles comprising an electrically-conductive material, and
functionalizing the surface of the conductive material with a binding reagent that binds to an analyte.

Clause 2: The method of clause 1, wherein the conductive material is deposited as a plurality of protuberances onto the surface of the substrate.

Clause 3: The method of clause 2, wherein the protuberances and have a diameter of not greater than 10 millimeters, and the area of the substrate comprising the protuberances is less than or equal to 200 square millimeters (mm²) and comprises at least one protuberance per mm².

Clause 4: The method of clause 2, wherein the area of the substrate comprising the protuberances is 4 mm² and comprises at least 9 protuberances per mm², at least 16 protuberances per mm², or at least 25 protuberances per mm² or the area of the substrate comprising the protuberances is 16 mm² and comprises at least 4 protuberances per mm² or at least 6.25 protuberances per mm².

Clause 5: The method of any one of clauses 1 to 4, wherein the conductive material is deposited as an ink comprising the particles, a binder, and/or solvents.

Clause 6: The method of clause 5, wherein the particles are nanoparticles or microparticles and/or are deposited by aerosol jet printing.

Clause 7: The method of clause 6, wherein the particles are nanoparticles having a diameter of at least 4 nanometers to not greater than 1 micron.

Clause 8: The method of clause 6, wherein the particles are microparticles having a diameter of at least 1 micron to not greater than 1 millimeter.

Clause 9: The method of any one of clauses 1 to 8, wherein the particles are suspended in a non-conductive polymer solution.

Clause 10: The method of any one of clauses 1 to 9, wherein the droplets comprise a solvent, and substrate is maintained at a temperature of 50° C. or greater during the deposition of the protuberances to evaporate the solvent.

Clause 11: The method of any one of clauses 1 to 10, wherein the electrically-conductive material of the particles comprises gold, silver, platinum, nickel, rhodium, zinc, an alloy of any of the preceding, carbon, a conductive polymer, graphene, such as graphene oxide, molybdenum disulfide (MoS₂), MXenes, such as titanium carbide, or any combination thereof.

Clause 12: The method of clause 11, wherein the electrically-conductive material of the particles comprises gold.

Clause 13: The method of clause 11, wherein the electrically-conductive material of the particles comprises a conductive polymer.

Clause 14: The method of any one of one of clauses 2 to 13, wherein the protuberances are individual pillars.

Clause 15: The method of clause 14, wherein the individual pillars have a height ranging from 1 micron to 1,000 microns and a diameter ranging from 0.1 microns to 500 microns.

Clause 16: The method of any one of clauses 2 to 13, wherein the protuberances form an open cell lattice.

Clause 17: The method of clause 16, wherein the open cell lattice comprises a plurality of unit cells, wherein each unit cell comprises a plurality of trusses joined at one or more joints and, together with one or more unit cells of the lattice, forming a repeated pattern of trusses defining at least a portion of the lattice.

Clause 18: The method of any one of clauses 1 to 17, further comprising sintering the deposited conductive material.

Clause 19: The method of clause 18, wherein the sintering is conducted at a temperature above 100° C. for at least 10 minutes.

Clause 20: The method of any one of clauses 1 to 19, further comprising coating the deposited conductive material with an electrically active material.

Clause 21: The method of clause 20, wherein the electrically active material comprises graphite, hard carbon, synthetic graphite, carbon black, graphene, such as graphene oxide, carbon nanotubes, gold, molybdenum disulfide (MoS₂), MXenes, such as titanium carbide, or any combination thereof.

Clause 22: The method of clause 21, wherein the electrically active material comprises graphene.

Clause 23: The method of any one of clauses 1 to 22, further comprising coating the deposited conductive material and/or electrically conductive material with a linking molecule comprising a first portion, a second portion, and a linking portion, wherein the first portion of the linking molecule comprises a functional group for attachment of the linking molecule to the surface of the protuberance, the second portion comprises a functional group for attachment of the linking molecule to the binding reagent, and the linking portion of the molecule extends between the first portion and the second portion.

Clause 24: The method of clause 23, wherein the linking molecule is (3-aminopropyl)triethoxysilane (APTES), L-Cysteine, thioglycolic acid, poly(ethylene glycol), N-hydroxysuccinimide esters, 11-mercaptoundecanoic acid, 12-mercaptodeodecanoic acid, or any combination thereof.

Clause 25: The method of any one of clauses 20 to 24, further comprising reacting the electrically active material or the second portion of the linking molecule with the binding reagent, to link the binding reagent to the deposited conductive material.

Clause 26: The method of any of one of clauses 1 to 25, wherein the binding reagent comprises: a protein, such as a lectin; an antibody or an antibody fragment; an epitope-containing polypeptide, an antigen; an aptamer, an affimer, a nucleic acid or any combination of the preceding.

Clause 27: The method of clause 26, wherein the binding reagent comprises an antigen or epitope of a pathogen, such as a virus, a bacteria, a fungus, or a parasite, such as a protein of a coronavirus, such as SARS-CoV-2, ebola virus, human immunodeficiency virus (HIV), influenza virus, herpes virus, zika virus, Escherichia coli, or Mycobacterium tuberculosis.

Clause 28: The method of clause 27, wherein the antigen is a SARS-CoV-2 spike protein antigen.

Clause 29: An electrode, comprising:
a substrate comprising a droplet-based printed, and optionally sintered, conductive material, and
a coating comprising a binding reagent covalently bonded to the surface of the conductive material that binds to an analyte.

Clause 30: The electrode of clause 29, wherein the deposited conductive material comprises gold, silver, platinum, nickel, rhodium, zinc, alloys of any of the preceding, carbon, a conductive polymer, graphene, such as graphene oxide, molybdenum disulfide (MoS₂), MXenes, such as titanium carbide, or any combination thereof.

Clause 31: The electrode of claim 29 or 30, wherein the conductive material is deposited as a plurality of protuberances onto the surface of the substrate.

Clause 32: The electrode of clause 31, wherein the area of the substrate comprising the protuberances is less than or equal to 200 square millimeters (mm²) and comprises at least 1 protuberance per mm².

Clause 33: The electrode of clause 31, wherein the area of the substrate comprising the protuberances is 16 mm² and comprises at least 4 protuberances per mm² or at least 6.25 protuberances per mm² or the area of the substrate comprising the protuberances is 4 mm² and comprises at least 9 protuberances per mm², at least 16 protuberances per mm², or at least 25 protuberances per mm².

Clause 34: The electrode of any one of claims 31 to 33, wherein the protuberances are individual pillars.

Clause 35: The electrode of clause 34, wherein the individual pillars have a height ranging from 1 micron to 1,000 microns and a diameter ranging from 0.1 microns to 500 microns.

Clause 36: The electrode of any one of clauses 31 to 33, wherein the protuberances form an open cell lattice.

Clause 37: The electrode of clause 36, wherein the open cell lattice comprises a plurality of unit cells, wherein each unit cell comprises a plurality of trusses joined at one or more joints and, together with one or more unit cells of the lattice, forming a repeated pattern of trusses defining at least a portion of the lattice.

Clause 38: The electrode of any of one of clauses 29 to 37, wherein the binding reagent comprises: a protein, such as a lectin; an antibody, an antibody fragment, or an engineered antibody, e.g., an scFv; an epitope-containing polypeptide, an antigen; an aptamer, a nucleic acid, or any combination of any of the preceding.

Clause 39: The electrode of clause 38, wherein the binding reagent comprises an antigen or epitope of a protein of a virus, a bacteria, a fungus, or a parasite, such as a protein of a coronavirus, such as SARS-CoV-2, ebola virus, human immunodeficiency virus (HIV), influenza virus, herpes virus, zika virus, Escherichia coli, or Mycobacterium tuberculosis.

Clause 40: The electrode of clause 39, wherein the antigen or epitope is a SARS-CoV-2 spike protein antigen or epitope.

Clause 41: A microfluidic test device comprising:
one or more sensing electrodes in a chamber or channel configured to receive a liquid test sample,
wherein the sensing electrode comprises a substrate and an electrode array comprising a working electrode, a counter electrode, and, optionally, a reference electrode on the substrate, and the working electrode comprises a droplet-based printed, and optionally sintered, conductive material, and
a coating comprising a binding reagent covalently bonded to the surface of the conductive material that binds to an analyte.

Clause 42: The microfluidic test device of clause 41, wherein the conductive material is deposited as a plurality of protuberances.

Clause 43: The microfluidic test device of clause 41 or 42, comprising:
a microfluidic channel;
an inlet flowing into the microfluidic channel;
an outlet flowing out of the microfluidic channel; and
the one or more sensing electrodes in direct contact with the microfluidic channel.

Clause 44: The microfluidic test device of any one of clauses 41 to 44, wherein the sensing electrode comprises a plurality of leads attached independently to the counter electrode, the working electrode, and, when present, to the reference electrode, and a module configured to send an electrical signal to and/or receive an electrical signal from the sensing electrode.

Clause 45: The microfluidic test device any one of clauses 41 to 44, wherein the test device comprises at least two sensing electrodes.

Clause 46: The microfluidic test device of any one of clauses 41 to 44, wherein the chamber or channel comprises soda-lime glass, polydimethylsiloxane, polymethyl methacrylate, sapphire, or any combination thereof.

Clause 47: The microfluidic test device of any one of clauses 41 to 46, wherein the substrate comprises glass, silicon, silicon dioxide, or any combination thereof.

Clause 48: The microfluidic test device of any one of clauses 41 to 47, wherein the microfluidic channel is configured to separate plasma from blood.

Clause 49: The microfluidic test device of any one of clauses 41 to 48, wherein the binding reagent comprises an antigen or epitope of a protein of a virus, a bacteria, a fungus, or a parasite, such as a protein of a coronavirus, such as SARS-CoV-2, ebola virus, human immunodeficiency virus (HIV), influenza virus, herpes virus, zika virus, Escherichia coli, or Mycobacterium tuberculosis.

Clause 50: The microfluidic test device of clause 49, wherein the antigen or epitope is a SARS-CoV-2 spike protein antigen or epitope.

Clause 51: The microfluidic test device of any one of clauses 41 to 50, comprising two different working electrodes, each having different binding reagents.

Clause 52: The microfluidic test device of clause 51, wherein a first working electrode comprises a SARS-CoV-2 antigen or epitope, and a second working electrode comprises an influenza antigen or epitope.

Clause 53: A method of sensing an analyte, the method comprising:
contacting a fluid comprising the analyte with the electrode of any one of clauses 29 to 40.

Clause 54: The method of clause 53, wherein the fluid is plasma.

Clause 55: The method of clause 53, wherein the analyte is a binding partner to the binding reagent.

Clause 56: The method of clause 55, wherein the analyte is an antibody.

Clause 57: The method of any one of clauses 53 to 56, wherein the binding reagent comprises an antigen or epitope of a virus, a bacteria, a fungus, or a parasite, such as a protein of a coronavirus, such as SARS-CoV-2, ebola virus, human immunodeficiency virus (HIV), influenza virus, herpes virus, zika virus, Escherichia coli, or Mycobacterium tuberculosis.

Clause 58: The method of clause 57, wherein the antigen or epitope is a SARS-CoV-2 spike protein antigen or epitope.

Clause 59: The method of any one of clauses 53 to 58, comprising two different working electrodes, each having different binding reagents.

Clause 60: The method of clause 59, wherein a first working electrode comprises a SARS-CoV-2 antigen or epitope, and a second working electrode comprises an influenza antigen or epitope.

### BRIEF DESCRIPTION OF THE DRAWINGS

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the invention.

FIG. 1 is a schematic of the manufacturing process of an embodiment of the current invention, i.e., a three-dimensional electrochemical sensor for rapid pathogenic biomarker detection. The sensor shown in FIG. 1 is a 3D printed COVID-19 test chip (3DcC). FIG. 1 (a) depicts a glass substrate with patterned gold film forming the base for working electrode (WE), counter electrode (CE) and reference electrode (RE) of the electrochemical cell of 3DcC. FIG. 1 (b) depicts the construction of the 3D printer (in this case an Aerosol Jet or AJ machine). FIG. 1 (c) depicts an AJ printed 10×10 gold micropillar array where an example pillar-to-pillar gap is indicated. FIG. 1 (d) depicts the details of AJ printing of a single micropillar where rapid layer-by-layer stacking of the micro-rings of the nanoparticle ink are achieved using surface tension (y) of the printed ink, without the use of any support structure. FIG. 1 (e) depicts the process of fabricating a polymer housing (in this case, polydimethylsiloxane or PDMS) of the 3DcC device. FIG. 1 (f) depicts the 3DcC device formed by placing the PDMS housing with microfluidic channel on the glass substrate with the micropillar electrodes. Prior to this step, the micropillar electrodes were functionalized with reduced graphene oxide (rGO) and viral antigens as described in FIGS. 2A-2C.

FIGS. 2A-2G depict the functionalization of 3D printed micropillar electrode (FIGS. 2A-2E) and 3DcC sensor principle/operation (FIGS. 2F and 2G). FIG. 2A depicts AJ printed gold micropillars prior to the surface treatment. FIG. 2B depicts the coating of the AJ printed gold micropillars by carboxylated (—COOH) rGO sheets by a simple drop-casting process, with a SEM image showing the decoration of rGO sheets on the gold pillar. The molecular structure of a rGO sheet is shown where —COOH and —OH groups are indicated. FIG. 2C depicts the coupling of the viral antigens with the rGO sheets using EDC:NHS chemistry. FIG. 2D depicts the antibodies selectively attached to the specific antigens upon introduction to the sensor via an antibody-antigen interaction. FIG. 2E shows schematic of a SARS-CoV-2 antibody attached to a micropillar electrode, with corresponding antigens attaching to the antibodies upon introduction to the sensor via an antigen-antibody interaction. FIGS. 2F and 2G are schematics showing the sensing principle of the 3DcC device. The electrode/electrolyte interface of the WE was expected to form an electrical double layer (C_{dl}), inner Helmholtz plane or IHP, outer Helmholtz plane or OHP, and a diffusion layer during the redox reaction. An equivalent electrical circuit is also shown. When antibodies are introduced in FIG. 2G, they rapidly bind with the antigens on the electrode surface, altering the Nyquist plot (schematics in FIGS. 2F and 2G) which is captured by electrical impedance spectroscopy (EIS).

FIGS. 3A-3F depict the physical and chemical characterizations of the 3DcC device. FIG. 3A is an optical image of the 3DcC device made by the fabrication process described in FIGS. 1 (a)-(f) and 2A-2C. FIG. 3B includes optical and SEM images of the AJ printed gold micropillar array (10×10) at different magnifications. FIG. 3C are SEM images showing the morphology of a single gold micropillar at different magnifications. FIG. 3D are SEM images showing the morphology of a gold micropillar after rGO decoration. FIG. 3E is Raman spectra of an AJ printed gold micropillars without a coating, after coating with rGO, and after immobilization of antigens on rGO-Au. The spectra show both defect and graphitic peaks for the coated samples. The graphitic peak is shifted to higher frequency upon S1 antigen immobilization. FIG. 3F is a photograph of the 3DcC device interfaced with a portable potentiostat which was connected to a smartphone via a USB-C connection to record the signal using PStouch software.

FIGS. 4A-4C are additional SEM images of AJ printed 3D gold micropillar electrodes of the 3DcC device. FIG. 4A includes SEM images of an electrode without any coating at different magnifications. The higher magnification image shows a characteristic surface porosity and roughness that aids in binding of the rGO sheets to the surface. FIG. 4B includes SEM images of the rGO coated electrode at different magnifications. The rGO forms a secondary three-dimensional structure as shown in the high magnification image. FIG. 4C includes SEM images of the rGO sheets attached to the surface of base Au layer. The overspray from the AJ printing process during micropillar formation creates surface features that aid in attaching the rGO sheets.

FIG. 5 depicts the geometry of the 10×10 micropillar array electrode, with dimensions. These dimensions are chosen to approximately match with that observed in the device of FIGS. 3A-3D.

FIGS. 6A-6D depict graphs showing the sensing of S1 antibodies and RBD antibodies. FIG. 6A is a graph of the magnitude of impedance for 3DcC device for sensing of S1 antibodies at a single point of time (12 s) plotted against the concentrations of spike S1 antibodies. FIG. 6B is a graph showing the times required for the signal to reach 93.2% of the saturated signal in seconds for 12 different sensors (6 for spike S1 antibodies and 6 for RBD antibodies). FIGS. 6C and 6D are graphs showing the sensor calibration plots for the detection of spike S1 and RBD antibodies, respectively. Three repeat readings were used at each concentration with 2× regenerations (i.e., a total of 9 data points per concentration). The R_{ct} values were obtained from the Nyquist plots in FIGS. 12A-12C and 14A-C. The sensitivity for both the devices has two ranges. For spike S1 antibody sensor, the slope of the curve is 0.27 kΩ/nM (1 fM-0.1 nM) and 4.5 kΩ/nM (0.1 nM-30 nM). For the RBD antibody sensor, the slope is 0.39 kΩ/nM (1 fM-1 nM) and 1.7 kΩ/nM (1 nm−20 nm).

FIGS. 7A-7C are energy-dispersive X-Ray (EDX) spectra of Au micropillars without any coating (FIG. 7A), with rGO coating (FIG. 7B), and after antigen immobilization on rGO-Au surface (FIG. 7C). Insets show the SEM images of the locations from where the spectra were obtained.

FIGS. 8A-8G depict electrochemical characterizations of the 3DcC Device. FIGS. 8A, 8B, and 8C are cyclic voltammograms of the device with bare 3D Au (AJ printed) and 2D Au (AJ printed) electrodes (FIG. 8A), same electrodes when coated with only rGO (FIG. 8B), and the same electrodes when coated with rGO and functionalized with SARS-CoV-2 spike S1 antigens (FIG. 8C). The scan rate of this study was set to 0.5 Vs within a fixed range of potential (+0.7V to −0.7V). FIGS. 8D and 8E are cyclic voltammograms depicting the scan rate studies of the sensors in FIG. 8C. The scan rate was varied from 0.5 to 2 V/s. FIG. 8F is a graph depicting the anodic and cathodic currents of the electrodes in FIGS. 8D and 8E plotted against the square root of the scan rate. FIG. 8G is a graph depicting the diffusion co-efficient calculated from Randles-Sevcik equation for different electrodes evaluated in FIGS. 8A-8C. All the measurements were conducted in presence of 50 mM phosphate buffer solution (PBS, pH 7.4) containing an equimolar concentration (5 mM) of ferro/ferricyanide [Fe(CN)₆]^{3-/4-}.

FIGS. 9A-9D depict electrochemical impedance spectroscopic (EIS) studies of AJ printed 3D Au and AJ printed 2D Au electrodes. FIG. 9A shows EIS measurements showing Nyquist plots of AJ printed 3D Au electrode without any coating, that coated with rGO, and that coated with rGO plus functionalized with SARS-CoV-2 spike S1 antigen. FIG. 9B shows EIS measurements showing Nyquist plots of AJ printed 2D Au electrode without any coating, that coated with rGO, and that coated with rGO plus functionalized with SARS-CoV-2 spike S1 antigen. The inset of FIG. 9B shows an equivalent circuit with elements such as charge transfer resistance (R_{ct}), double layer capacitance (Cal), Warburg resistance (Z_{w}), and solution resistance (Rₛ). FIG. 9C shows the phase shift (θ) plots for the electrodes with AJ printed 3D Au and AJ printed 2D Au electrodes coated with rGO only and coated with rGO plus spike S1 antigens. FIG. 9D is a comparison plot of phase shift for the AJ printed 3D Au and AJ printed 2D Au electrodes. All EIS measurements were conducted in the presence of 50 mM phosphate buffer solution (pH 7.4) containing an equimolar concentration (5 mM) of ferro/ferricyanide [Fe(CN)₆]^{3-/4-}.

FIGS. 10A-10F are graphs depicting the COMSOL simulations showing the effect of the pillar-to-pillar distance, t, pillar height, h, and pillar diameter, d, on the electrochemical performance of the 3DcC device. FIGS. 10A and 10B show the effect of the configuration of the pillar array (and therefore pillar-to-pillar distance) on total current and saturation current, respectively. In these calculations, the area of the working electrode (2 mm×2 mm) was kept constant. The 10×10 array configuration chosen in this work is indicated in FIG. 10B. FIGS. 10C and 10D show the effect of the pillar height, h, on total current and saturation current, respectively. The range of experimental variation observed is indicated in FIG. 10D. The pillar diameter for this simulation was kept at 120 μm and the total area of the working electrode was 2 mm×2 mm FIGS. 10E and 10F show the effect of the pillar diameter, d, on total current and saturation current, respectively. The range of experimental variation observed is indicated in FIG. 10F. The pillar height for this simulation was kept at 75 μm and the total area of the working electrode was 2 mm×2 mm.

FIGS. 11A-11D are graphs depicting the sensing of Antibodies to SARS-CoV-2 Spike S1 Antigen at Different Molar Concentrations with Regeneration. FIG. 11A are Nyquist plots of the 3DcC sensor measured by EIS method without and with the spike S1 antibodies at concentrations of 0.01 fM, 1 fM, 1 pM, 100 pM, 1 nM, 10 nM, and 30 nM in PBS buffer solution. FIGS. 11B and 11C are Nyquist plots similar to that in FIG. 11A after two successive sensor regenerations by a low pH chemistry consisting of 1.0 M (pH 2.5) formic acid solution. The regeneration was achieved within 60 seconds. For all concentrations, the signal in FIGS. 11B and 11C was within 95% of that in FIG. 11A. FIG. 11D depicts the charge transfer resistance (R_{ct}) for the 3DcC sensor for each concentration of antibodies and control serum before and after each regeneration for the data in FIGS. 11A-11C. The fetal bovine serum (fbs) and rabbit serum (rs) in FIGS. 11A-11D were utilized as control biofluids. For all measurements, a 50 mM PBS (pH 7.4) solution containing an equimolar concentration (5 mM) of a ferro/ferricyanide mediator was used. Three successive readings were obtained at each concentration of the antibodies for the three data-sets. Detection frequencies from 1 Hz to 10,000 Hz were applied to obtain this data. There was no incubation time for all the measurements in this figure. The R_{ct} values in FIG. 11D were calculated by fitting the data in FIGS. 11A-11C to a Randles equivalent circuit shown in FIG. 2F.

FIGS. 12A-12H are graphs depicting two additional data sets for sensing of SARS-CoV-2 spike S1 and RBD antibodies at different molar concentrations with regeneration. FIGS. 12A and 12C are Nyquist plots of the two 3DcC sensors without and with the spike S1 antibodies at concentrations of 0.001, 0.01, 0.1, 1, 10, 100, 1000, and 30000 pM in PBS buffer solution. FIGS. 12B and 12D are the R_{ct} values for the data represented in FIGS. 12A and 12C, respectively. FIGS. 12E and 12G are Nyquist plots of the two 3DcC sensors without and with the spike RBD antibodies at concentrations of 0.001, 0.01, 0.1, 1, 10, 100, 1000, and 10,000 pM in PBS buffer solution. FIGS. 12F and 12H are the R_{ct} values for the data represented in FIGS. 12E and 12G, respectively. A formic acid assay (1.0 M; pH 2.5) was used for regeneration which was carried out in 60 seconds.

FIGS. 13A-13D are graphs depicting the sensing of Antibodies to SARS-CoV-2 receptor binding domain (RBD) Antigens at Different Molar Concentrations with Regeneration. FIG. 13A are Nyquist plots of the 3DcC sensor measured by EIS method without and with the RBD antibodies at concentrations of 0.01 fM, 1 fM, 1 μm, 100 pM, 1 nM, 10 nM, and 30 nM in PBS buffer solution. FIGS. 13B and 13C are Nyquist plots similar to that in FIG. 13A after two successive sensor regenerations by a low pH chemistry consisting of 1.0 M (pH 2.5) formic acid solution. The regeneration was achieved within 60 seconds. For all concentrations, the signal in FIGS. 13B and 13C was within 95% of that in FIG. 13A. FIG. 13D depicts the charge transfer resistance (R_{ct}) for the 3DcC sensor for each concentration of antibodies and control serum before and after each regeneration for the data in FIGS. 13A-13C. The fetal bovine serum (fbs) and rabbit serum (rs) in FIGS. 13A-13D were utilized as control biofluids. For all measurements, a 50 mM PBS (pH 7.4) solution containing an equimolar concentration (5 mM) of a ferro/ferricyanide mediator was used. Three successive readings were obtained at each concentration of the antibodies for the three data-sets. Detection frequencies from 1 Hz to 10,000 Hz were applied to obtain this data. There was no incubation time for all the measurements in these figures. The R_{ct} values in FIG. 13D were calculated by fitting the data in FIGS. 13A-13C to a Randles equivalent circuit shown in FIG. 2F.

FIGS. 14A-14B are dose-response plots of data from FIG. 11D (FIG. 14A) and data from FIG. 13D (FIG. 14B). The minimum R_{ct} values required for sensing are identified.

FIGS. 15A-15F depict the detection time and regeneration studies of the 3DcC Device. FIGS. 15A and 15B depict the detection time for spike S1 and RBD antibodies during the EIS measurements, respectively, for twelve different 3DcC devices. The device impedance is plotted against the detection time. The spike S1 and RBD sensors reached 93.2% and 92% of their saturation impedance at 11.5 seconds, respectively, allowing the signal detection in seconds. The concentration of antibodies was set to 1 nM for this measurement. A frequency range of 1-10000 Hz was used to obtain this data. The (Zᵢₘ) data is recorded from 3 seconds after the introduction of antibodies as the EIS measurement had to overcome the solution resistance, Rₛ prior to obtaining the charge transfer resistance (see schematic in FIG. 2F). FIGS. 15C and 16D depict regeneration studies showing Nyquist plots and charge transfer resistance for the detection of spike S1 antibodies for the 3DcC device. The regeneration is carried out 9-times (a total of 10 readings). For each measurement, the sensor was first exposed to PBS, then to 1 nM concentration of S1 antibodies in PBS, and finally to formic acid (1M) with an incubation time of 60 s. The charge transfer resistance as a function of the number of regenerations is plotted in FIG. 15D. FIGS. 15E and 15F show the regeneration data for sensing of RBD antibodies by the 3DcC device. For both the sensors, a minimal loss in sensor performance is observed after 9 regenerations.

FIGS. 16A-16H are graphs depicting the cross-reactivity and reproducibility studies of the 3DcC Device. FIG. 16A is a graph showing the cross-reactivity test for the 3DcC device designed to detect spike S1 antibodies. Nyquist plots for the device are plotted for multiple antigens and antibodies in absence and presence of spike S1 antibodies. Interleukin-6 or IL-6 antigen (0.05 nM), nucleocapsid (N) antibody (1 nM), and RBD antibody (0.1 nM) are used for this measurement. FIG. 16B is a graph depicting the R_{ct} values that were calculated from the Nyquist plots shown in FIG. 16A. The 3DcC device provided minimal interference with other related proteins. FIG. 16C is a graph showing the cross-reactivity test for the 3DcC device designed to detect RBD antibodies. Nyquist plots for the device are plotted for multiple antigens and antibodies in absence and presence of RBD antibodies. Interleukin-6 or IL-6 antigen (0.05 nM), nucleocapsid (N) antibody (1 nM), and spike S1 antibody (0.1 nM) are used for this measurement. FIG. 16D is a graph depicting the R_{ct} values that were calculated from the Nyquist plots shown in FIG. 16C. The 3DcC device provided minimal interference with other related proteins. FIGS. 16E and 16F depict the sensor reproducibility test on six different 3DcC sensors in presence of S1 antibodies (1 nm in PBS). The sensor-to-sensor variation is evaluated by calculating the R_{ct} values for each sensor. This variation is within about 6%. The error bar is from at least three repeated measurements of the sensor. FIGS. 16G and 16H are graphs depicting the reproducibility test data for sensing of RBD antibodies (1 nM in PBS) from six different sensors. This sensor-to-sensor variation in this case was about 5%.

FIGS. 17A-17D show the kinetics of antigen-antibody interactions. FIGS. 17A-17B are Nyquist plots for spike S1 antibodies of concentration 10 nM (FIG. 17A) and 30 nM (FIG. 17B) at the micropillar surface indicating their association, equilibrium, and regeneration. FIG. 17C shows the variation in the charge transfer resistance (R_{ct}) during antigen-antibody interaction for spike S1 antibodies with 10 nM and 30 nM concentration. Repeat 1, 2, 3, and 4 are the replicate plots in the equilibrium phase. FIG. 17D is a schematic of the association, equilibrium, and regeneration of the kinetics shown in FIGS. 17A-17C. In associate phase, the target antibodies (10 and 30 nM) with buffer solution are loaded into the 3DcC device. The antibodies attach to the antigens. However, some unbound antibodies may be present in the solution near the sensor surface and contribute to the R_{ct}. In equilibrium phase, a fresh PBS (without antibodies) is used to wash the 3DcC device, thus removing the unbound antibodies in the solution, and reducing the R_{ct} slightly. In the regeneration phase, a solution of formic acid (1 M; pH 2.5) is used to regenerate the sensor surface, thus eluting the antibodies from the antigens and lowering R_{ct} significantly.

FIGS. 18A and 18B are graphs depicting an additional study of the kinetics of antigen-antibody interactions using two different sensors for detection of spike RBD antibodies. FIGS. 18A and 18B show the variation in R_{ct} during antigen-antibody interaction during association, equilibrium, and replicates (repeat 1 and repeat 2) for 1 nM and 10 nM concentration of spike RBD antibody, respectively. Insets show the corresponding Nyquist plots.

FIGS. 19A-19C are graphs depicting the sensing of antigens to SARS-CoV-2 Spike S1 antibodies at different molar concentrations with regeneration (FIG. 2E depicts the schematic of the antigen detection mechanism). FIG. 19A is Nyquist plots of the 3DcC sensor measured by EIS method with the antigens at concentrations of 1 fM, 10 fM, 100 fM, 1 pM, 10 pM, and 100 pM in PBS buffer solution. FIG. 19B is a graph showing the charge transfer resistance (R_{ct}) with respect to the concentration of spike S1 antigens. FIG. 19C is a graph showing the R_{ct} as a function of the concentration of spike S1 antigens with two regeneration steps.

FIGS. 20A-20C are graphs depicting the sensing of antibodies from human serum samples. FIG. 20A is a graph showing that antibodies were detected from human serum samples that had a positive COVID-19 test. FIGS. 20B and 20C are the plots of impedance components such as real (Zᵣₑ) and imaginary (Zᵢₘ) impedances with detection time in presence of human spiking plasma with their serial dilution. In this spiking plasma analysis, a 10 nM of RBD antibody is added in a set of human plasma concentrations. These examples indicate that the sensor showed a distinguished single in presence of RBD antibodies in different real human plasma concentrations.

FIG. 21 depicts exemplary open cell lattice structures that can be used as electrodes.

FIG. 22 is a schematic drawing showing the functionalization of silver protuberances coated with gold with a binding reagent.

### DESCRIPTION OF THE INVENTION

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. Further, as used herein, all numbers expressing dimensions, physical characteristics, processing parameters, quantities of ingredients, reaction conditions, and the like, used in the specification and claims are to be understood as being modified in all instances by the term “about”. Moreover, unless otherwise specified, all ranges disclosed herein are to be understood to encompass the beginning and ending range values and any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 3.3, 4.7 to 7.5, 5.5 to 10, and the like.

As used herein “a” and “an” refer to one or more. The term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The terms “a” and “an” are intended to refer to one or more.

As used herein, spatial or directional terms, such as “left”, “right”, “inner”, “outer”, “above”, “below”, “over”, “under”, and the like, relate to the invention as it is shown in the drawing figures are provided solely for ease of description and illustration, and do not imply directionality, unless specifically required for operation of the described aspect of the invention. It is to be understood that the invention can assume various alternative orientations and, accordingly, such terms are not to be considered as limiting.

A “group” or “functional group” is a portion of a larger molecule comprising or consisting of a grouping of atoms and/or bonds that confer a chemical or physical quality to a molecule. A “residue” is the portion of a compound or monomer that remains in a larger molecule, such as a polymer chain, after incorporation of that compound or monomer into the larger molecule. A “moiety” is a portion of a molecule, and can comprise one or more functional groups.

To prepare electrode assemblies and a three-dimensional (3D) electrode array as described herein, a plurality of protuberances are deposited onto the surface of a substrate by a droplet-based printing method. In the droplet-based printing method, such as aerosol jet (AJ) printing, an electrically-conductive material is dispersed in a liquid medium, such as a solvent, and is deposited in a suitable pattern on a suitable substrate. An “array” refers to, in the context of the electrodes described herein, a substrate with multiple protuberances deposited on the surface of a substrate, e.g., by aerosol jet printing.

As used herein, “aerosol jet printing” or “AJ printing”, also referred to as Maskless Mesoscale Materials Deposition or M3D, involves atomization of ink, e.g., by ultrasound or by pressurized gas, and entraining the ink droplets into a stream of gas for delivery to a print head that focuses the gas stream, for example using a gas sheath. An aerosol jet printer, or AJ printer is a device or system used for aerosol jet printing. Aerosol jet printing is capable of producing and accurately-depositing ink particles of 10 microns (μm, or micrometers) or less. As such, aerosol jet printing is capable of producing structures/features 10 μm or greater in size. Aerosol jet printing is capable of delivering suitably-sized particles, such as particles having a diameter of not greater than 10 millimeters (mm), for preparation of electrode structures as described herein.

The particles may be nanoparticles or microparticles. By “nanoparticle(s)” it is meant particles in a size range, either absolute or statistically defined (e.g., average or median), of from 1 nm to 1000 nm, or more typically from 1 nm to 100 nm, which may be defined according to any standard, e.g., ultrafine particles or as defined under ISO/TS 80004. The particles may be nanoparticles having a diameter of at least 4 nanometers (nm) to not greater than 1 μm. The particles may be microparticles having a diameter of at least 1 μm to not greater than 1 mm.

An AJ printer creates an aerosol mist of the droplets of ink comprising the particles comprising an electrically-conductive material from a reservoir by using either a pneumatic or an ultrasonic atomizer and utilizes an aerodynamic focus to deposit aerosolized materials onto the substrate. Pneumatic atomization is used for the printing of thicker liquids such as polymers. The aerosol jet printing may be carried out with an atomizer gas flow rate of 1-30 sccm (standard cubic centimeters per minute) and a sheath gas flow rate of 1-70 sccm, which varies with particular liquid media and viscosities.

The AJ printing “ink” comprises particles suspended in a solvent. The solvent may be any suitable solvent, for example and without limitation: deionized water, ethylene glycol, toluene, hexane, 2-methoxyethanol, glycerol, 2-amino-2-methyl-1-propanol (AMP), tetradecane, or a combination of two or more of the preceding liquids. The solution may comprise a rheology modifier, such as ethylene glycol, N-vinylpyrrolidone, or hydrophobically modified ethylene oxide urethane (HEUR), or a combination of two or more rheology modifiers. The solvent and rheology modifier may be the same, as is the case of ethylene glycol. The ink may also comprise a binder or binding agent. Useful binding agents for metal particles, such as in the context of conductive ink include, without limitation: polyalkylene carbonates, acrylic resins, or 2-methoxyethanol, or a combination of two or more binding agents.

The particles for deposition by aerosol jet printing may be electrically-conductive materials such as a metals, carbon black, carbon allotropes (e.g., conductive carbon allotropes, such as graphite, carbon nanotubes, graphene, or fullerenes), graphene, such as graphene oxide, molybdenum disulfide (MoS₂), MXenes, such as titanium carbide, ceramics, conductive polymers, or any combination thereof. The particles may be metal, such as gold, silver, platinum, nickel, rhodium, zinc, an alloy of any of the preceding, or a combination of any of the preceding. The particles may be coated with a polymer, such as poly(ethylene glycol) (PEG), polyethylenimine, thiols, or amines, or a combination of any of the preceding, to inhibit agglomeration in the dispersion. The electrically-conductive particles may be suspended in a non-conductive polymer. The electrically-conductive particles may be droplets of a conductive polymer, such as, without limitation, a polyaniline, a polyacetylene, a polythiophene, a poly(p-phenylene sulfide), a poly(p-phenylene vinylene), a polyindole, or a polypyrrole, or a combination of any of the preceding.

Each protuberance of an array may be of the same or different materials or a combination of materials. If the printed protuberances are of different materials, the material is either mixed prior to printing, or printed independently, e.g., sequentially.

The protuberances may be formed by depositing, for example and without limitation by aerosol jet printing, onto a surface of a substrate a plurality of layers of the electrically conductive material. Non-limiting examples of suitable substrates include flexible or rigid polymers, metals, alumina, ceramics, silicon structures, glass, diodes, integrated circuit, or a circuit board such as a printed circuit board (PCB). Examples of flexible polymers include but are not limited to polydimethylsiloxane (PDMS), Kapton® (polyimide), or Poly(lactic acid) (PLA). The substrate also may include an electrical path, such as conductive leads, conductive traces, or conductive pads, for the electrodes to an external circuit. The protuberances may be deposited on or in electrical contact with a conductive lead, trace, or pad. Conductive traces, leads, or pads may be formed on a non-conducting substrate by methods such as physical vapor deposition or chemical vapor deposition with a thin layer of conducting material, such as a metal or a conducting polymer, to provide the electrical path. For example, the substrate may be a glass, coated with chromium and/or gold. Examples of suitable metal substrates include stainless steel, copper, aluminum, silver, gold, chromium, and tin. Metal substrates may optionally be coated or patterned with an additional, different conducting material and/or insulating layers, such as parylene, to form suitable electrical paths for the individual electrodes. For example, the substrate may be stainless steel, optionally with a 50 nm chromium coating or a thin poly(3,4-ethylenedioxythiophene) (PEDOT) coating. The substrate may be a component of a sensing electrode or other structure or device the array is to be incorporated into. For an electrode assembly, the substrate shape, and configuration or arrangement of the electrodes and 3D electrode arrays may be varied, depending on the ultimate structure of the assembly and any associated microfluidics.

The protuberances may be printed on a substrate that is planar or non-planar. The substrate may be selected to withstand heating to a sintering temperature of the material(s) forming the protuberances.

The area of the substrate having the printed protuberances may be less than or equal to 200 square millimeters (mm²), less than or equal to 100 mm², less than or equal to 50 mm², or less than or equal to 16 square millimeters (mm²), for example 100 mm², 75 mm², 50 mm², 25 mm², 20 mm², or 16 mm², or any increment in the stated ranges. The printed protuberance may have any suitable density, for example and without limitation, of at least one protuberance per mm², such as at least 2.25 protuberances per mm². For example, the area of the substrate having the protuberances may be 16 mm² and may have at least 4 protuberances per mm² or at least 6.25 protuberances per mm². The area of the substrate having the protuberances may be 4 mm² and may have at least 9 protuberances per mm², such as at least 16 protuberances per mm², or such as at least 25 protuberances per mm². Suitable densities of the protuberances may be optimized based on the thickness of the protuberances, providing sufficient gaps between the protuberances for liquid flow, for example and without limitation to maximize liquid flow through the array, and surface area of the protuberances. Suitable densities of the protuberances on the substrate may be optimized to control the limit of detection (LOD) and the electrical response of the 3D electrode array and can be tailored by one having ordinary skill in the art.

The protuberances can be made using other suitable additive manufacturing methods such as, but not limited to, inkjet printing, gravure printing, extrusion printing, and 2-photon lithography. In one example, the protuberances may be made using manufacturing methods such as, but not limited to, lithographic methods.

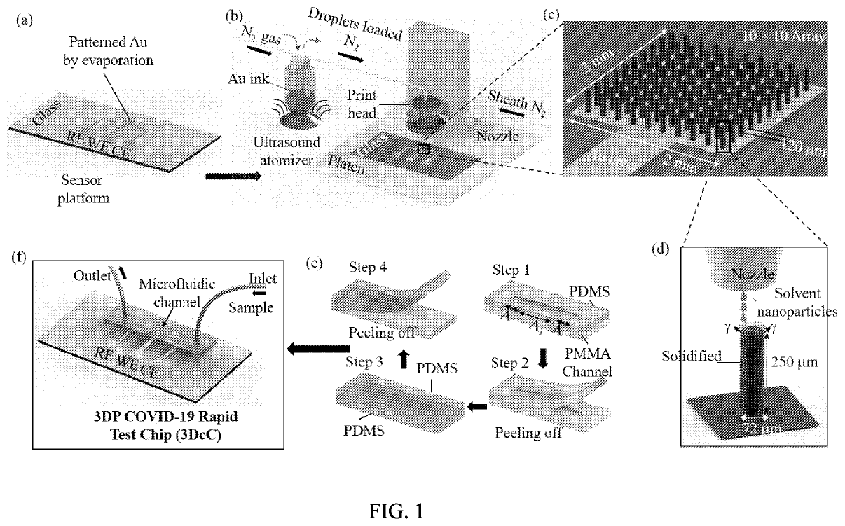

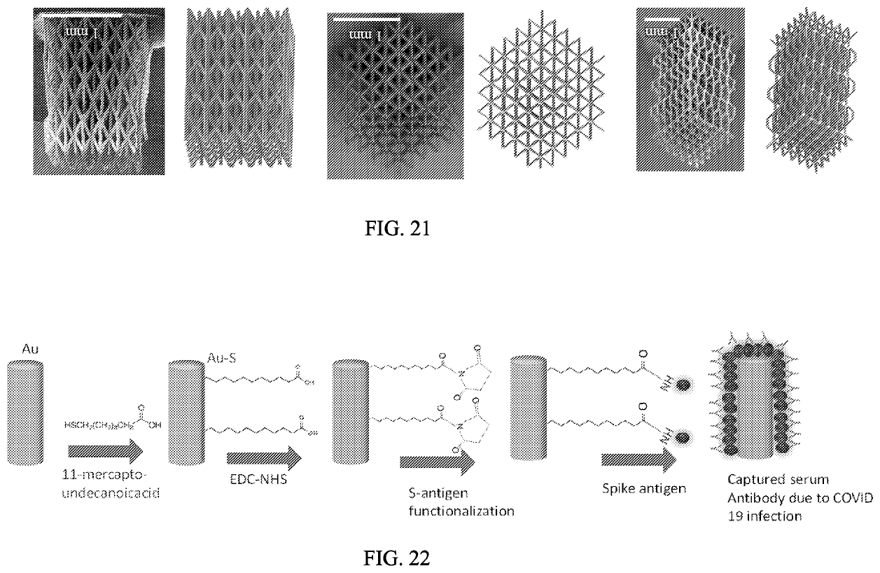

The ink from which the protuberance structures are fabricated may be dispensed, e.g., using an AJ printer, in multiple layers. The process of printing a 3D electrode array may involve deposition of one layer or droplet(s) of the particle solution followed by the use of heat or other form of energy to remove (evaporate) the solvent of the ink. The substrate, and therefore the protuberances may be heated to a temperature sufficient to remove the solvent by either heating the substrate to a suitable temperature, or by directing a laser at the site of particle solution deposition. The substrate temperature may be maintained in a range of from, for example and without limitation, 25° C. to 150° C. or increments there between, such as 80° C., 100° C., 110° C., or 125° C. After deposition and removal of solvent, a layer is formed, which by itself or in combination with other layers, e.g., in a layer of a protuberance, forms a solid base to receive the next printed layer. The next printed layer may have the same or different composition as the previously deposited layer. This process is repeated as desired, e.g., according to a predetermined protuberance configuration, to produce high aspect ratio, and high surface area electrodes, for example as shown in FIGS. 1 (b) and 1(c) and FIG. 21, without the need for any support material.

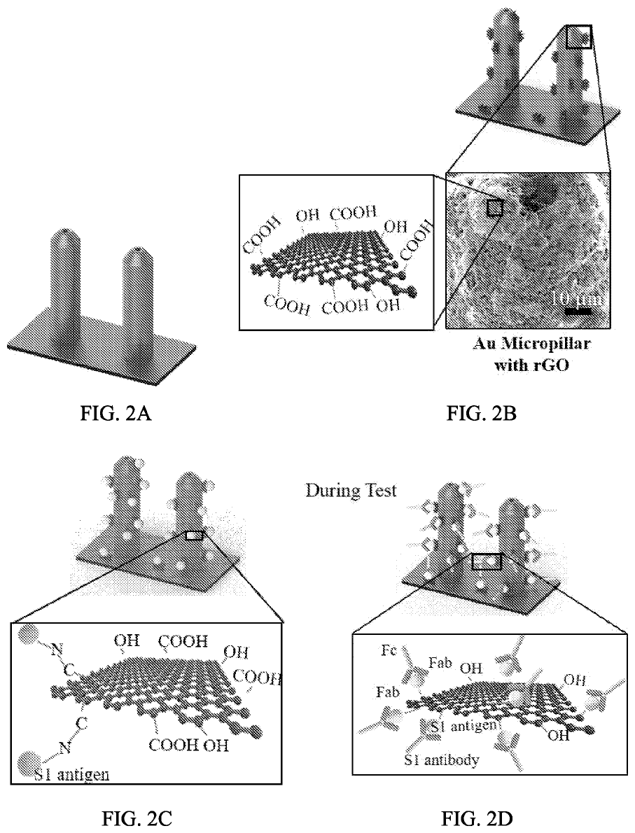

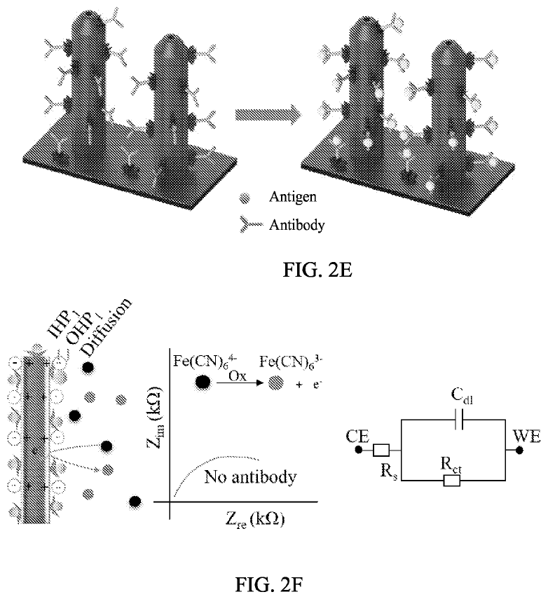

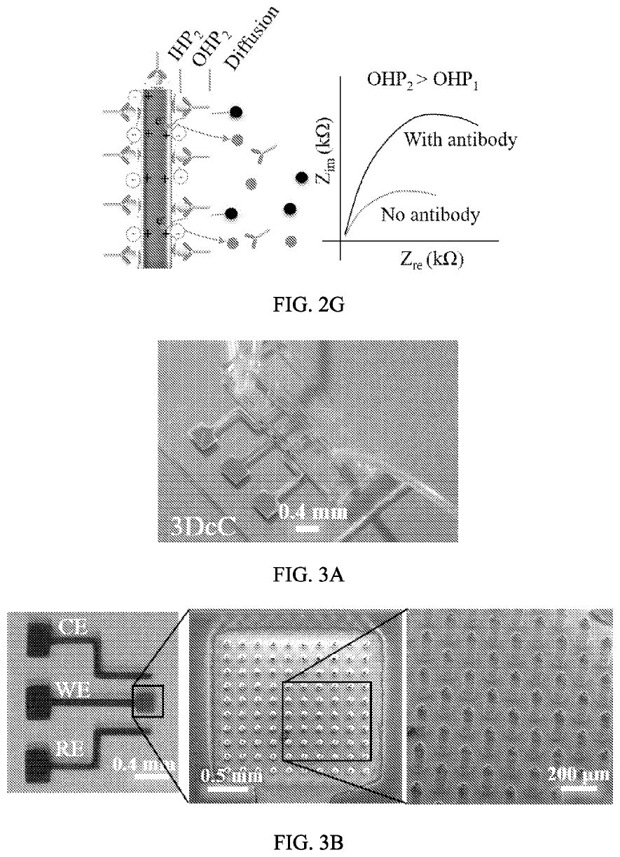

The protuberances may be deposited as individual pillars across an area of the substrate to form a 3D electrode array. By individual, it is meant that the pillars do not connect or bridge aside from their connection via the substrate, for example as compared to an open-cell matrix or lattice as described herein. The individual pillars may be solid. The individual pillars may be hollow (see, e.g., FIG. 2A). The solid individual pillars may have circular or rectangular or any other cross-section shape. The hollow individual pillars may have circular or rectangular or any other cross-section shape for the outer or the inner perimeter. The individual pillars may have a height ranging from 1 μm to 1,000 μm, including any increment there between, such as 50 μm, 75 μm, 100 μm, 125 μm, 150 μm, 175 μm, 200 μm, 225 μm, 250 μm, 250 μm, 275 μm, 300 μm, 350 μm, 400 μm, or 500 μm. The individual pillars may have a diameter ranging from 0.1 μm to 500 μm, including any increment there between such as 10 μm, 20 μm, 30 μm, 40 μm, 50 μm, 60 μm, 70 μm, 80 μm, or 90 μm. The distance between each of the individual pillars, or the pillar-to-pillar gap, may be from 2 μm to 500 μm, such as 20 μm to 400 μm, or 30 μm to 300 μm. The protuberances may be deposited onto the substrate in any shape, such as in the shape of a square or rectangle. The protuberances may be deposited on a substrate in the shape having an array dimension of 6×6, 8×8, or 10×10. The protuberances may be irregular or regular three-dimensional structures of truss, plate, or other shapes on the electrode surface. An example of lattice shaped three-dimensional protuberances is depicted in FIG. 21.

For example, the array dimension may be 10×10, the area of the substrate having the individual pillars may be 4 mm² and may have at least 25 individual pillars per mm².

Alternatively, at least a part of the protuberances may be deposited in the form of irregular or regular three-dimensional structures of trusses, plates, open cell lattice, or other shapes on the substrate. An open cell lattice comprises a plurality of unit cells, where each unit cell comprises a plurality of trusses joined at one or more joints. Together with one or more unit cells of the lattice, a repeated pattern of trusses defines at least a portion of the lattice. Exemplary lattice structures are shown in FIG. 21. A “lattice” is a regular geometrical arrangement of points or objects over an area or in space. In the context of the present disclosure, a lattice is a regular arrangement of trusses to form the lattice. The trusses may be rods that interconnect in a pattern to form an open lattice, as is depicted in FIG. 21. In some cases, the rods may be at least partly replaced with plates to make a plate lattice. The trusses are arranged to define a void, or space, between adjacent trusses. In an open lattice, the voids between trusses define open channels through the lattice, permitting fluid to flow through the lattice. The voids and open channels can have any suitable shape and configuration.

The trusses can be any suitable shape, so long as the arrangement thereof in the lattice permits flow of liquid throughout the lattice. Non-limiting shapes include: a rod, a cylinder, a column shape, a cylindroid, a scutoid, a conical shape, a polyhedron, a sphere, a spheroid, an ovoid, a spiral, or a helix. Shapes can be combined in order to make the lattice. The open lattice structures can be made of unit cell or cells of arbitrary shapes and sizes comprising two or more trusses joined at a joint or node in a geometric configuration. The smallest group of trusses in at least a portion of the lattice is a unit cell. The open lattice can have repeating or non-repeating unit cells. The number of repeating unit cells of the lattice structure may be any number, such as in the range of 1 to 100,000 in the X-dimension, Y-dimension, and/or Z-dimension.

Non-limiting examples of suitable repeated unit geometries include: square, rectangular, triangular, hexagonal, octahedral, rhomboidal, icosahedral, spherical, or any other regular or irregular shape and/or pattern of joined trusses. In one example, the open lattice has an octahedral unit cell comprising eight trusses joined to produce an eight-sided structure, formed from eight trusses joined at six nodes or joints. As would be understood by a person of ordinary skill, the stated geometric designation for any unit cell describes the arrangement of the trusses, and due to the three-dimensional shapes of the trusses, does not define absolutely a resultant 3D structure created by the trusses, for example because the trusses are cylindrical and therefore cannot form a perfect geometric edge (a line) for a shape such as an octahedron. Further, the trusses do not necessarily define every edge of a structure. The open lattice may have a combination of two or more unit cell geometries. For example, the open lattice can have a combination of hexagonal and octahedral unit cells. The open lattice may have different unit cells of the same or different sizes, where the cell size of the open lattice controls the porosity, in terms of voids and open channels. The cells may have a periodicity (average distance between centers of, or like features of adjacent unit cells) ranging from 1 μm to 1 mm, such as from 2 μm to 500 μm, from 10 μm to 1000 μm, or from 100 μm to 300 μm. The structure of the lattice may be such that individual cells are indistinguishable in a dimension, e.g., forming a tubular, or elongated cell, and periodicity may be only in one or two dimensions.

The trusses may be deposited perpendicular (normal), at a 90 degree (90°) angle to, a plane of the substrate, or at any other angle to the substrate between 0° and 90°, including any increment there between, such as 5°, 10°, 15°, 20°, 25°, 30°, 35°, 40°, 45°, 50°, 55°, 60°, 65°, 70°, 75°, 80°, or 85°. One or more of the trusses may be deposited normal to the substrate. One or more of the trusses may be deposited at an angle between 10° and 90°, such as between 30° and 40°, to the substrate. The trusses can be 3D printed straight, curved or any other shape suitable for the end use. The trusses can have different shapes, even within a single unit cell.

The open cell lattice comprises trusses joined at nodes, or joints, between truss structures, such as rods. Each lattice truss ranges from 1 μm to 1 mm in a major dimension (length, that is distance between nodes or joints), depending on the geometry of the lattice, to yield suitable cell periodicity in the range of from 2 μm to 500 μm, and in a lattice structure, trusses can be of the same length or a combination of lengths. For example, the lattice structure may contain trusses that are a single length throughout the lattice structure, or different lengths, to produce certain geometries. Lattice structures may have a length ranging from 5 μm to 5 centimeters (cm), such as from 100 μm to 1 cm or from 200 μm to 500 μm. The overall size and shape of the lattice may be selected to fit within the area of the electrode. The truss diameter, e.g., the diameter of a spherical truss, or a non-major dimension, such as a diameter of a rod, with a circular or ovoid cross-section, may range from 0.1 μm to 500 μm, such as 2 μm to 500 μm, such as 20 μm to 50 μm, such as 1 μm to 100 μm, and can be selected to optimize surface area and liquid flow through the matrix of the open cell lattice.

The as-printed protuberances may be sintered or joined together by some energy source, such as laser, UV light, or thermal heat. In some examples, the printed protuberance structures are not sintered, leaving the protuberance structures in a particle format. Sintering is the process of forming a solid mass of material by heat or pressure without melting it to the point of liquefaction. Sintering occurs naturally in mineral deposits, or as a part of a manufacturing process used with metals, ceramics, plastics, and other materials. In the context of the deposited particles described herein, particularly the metal particles, sintering is a heat treatment applied to a particle powder structure in order to impart strength and integrity. The temperature used for sintering is below the melting point of the major metal constituent of the particle material. After printing, neighboring powder particles are held together by cold welds, which give the compact sufficient “green strength” to be handled. At sintering temperature, diffusion processes cause necks to form and grow at these contact points. As a consequence of the sintering process, water and other liquid medium or solvents, rheology modifiers, and anti-agglomeration coatings are removed by evaporation and burning, and any surface oxides are reduced.

The particles comprising an electrically-conductive material, such as metal particles, printed on the surface of the substrate as a protuberance, may be sintered by raising the temperature to a temperature below the melting point of the metal particles. During the sintering process, the temperature may be raised at any rate. The temperature may be raised at a rate of less than 5° C. per minute, such as 1° C. per minute. Once the sintering temperature is reached, the sintering process may be stopped by reducing the temperature at any suitable rate, or the maximum sintering temperature may be maintained for any suitable time period, such as for 5 minutes (5′), 10′, 20′, 30′, one hour, 2 hours, or longer, including increments there between. The particles comprising an electrically-conductive material may be sintered by heating to temperatures that varies with the material. For example, sintering for gold nanoparticles occur in a temperature range of from about 200° C. to 500° C. Optimal maximum, sintering temperatures, temperature ramp rates, or durations may be empirically determined depending on the composition and structure of the particles.

Sintering the printed protuberances introduces hierarchical porosity. The hierarchical porosity can be controlled by varying the sintering temperature, duration, and temperature ramping rates. For example, the porosity of the protuberances may be from 20% to 30%.

An electrically active material may be deposited over at least a portion of the protuberances to form an outer shell. The protuberances may be coated, at least in part, with an electrically active material through an appropriate deposition method such as electroplating, atomic layer deposition (ALD), sputtering, physical vapor deposition, or chemical vapor deposition. Non-limiting examples of electrically active materials include conductors, semiconductors, insulators, or any combination thereof, such as, for example and without limitation: graphite, hard carbon, synthetic graphite, carbon black, graphene flakes (planar or 2D graphene, which alternatively may be referred to simply as graphene), carbon nanotubes, graphene oxide, MoS₂, MXenes, such as titanium carbide, or gold. For example, the protuberances may be gold and may be coated with graphene, e.g., graphene flakes. For example, the protuberances may be silver and may be coated with gold.

A linking molecule having a first portion, a second portion, and a linking portion may be deposited over at least a portion of the protuberances to link a binding reagent or binding partner to the protuberances. The protuberances may be in any form or composition such as the protuberances described above, e.g., the sintered protuberances or the protuberances coated with an electrically active material. The linking molecule having a first portion, a second portion, and a linking portion may have the following formula I:

X-Y-Z (I)

where X is the first portion, Z is the second portion, and Y is the linking portion that extends between the first portion and the second portion. X and Z can be functional groups that may be used to link a protein, an enzyme, a nucleic acid, or another suitable composition to the protuberances. For example and without limitation, X may be a thiol (—SH), an amine, such as a primary amine (—NH₂), a carboxylic acid (—C(O)OH), an aldehyde (—C(O)H), a hydroxyl (—OH), a phosphate (PO₄⁻), a sulfonic acid (—S(O)₂—OH), an ester (—C(O)O—), a cysteine moiety, or any functional group or any combination thereof that reacts with the electrically active material. Z may be, without limitation, a carboxylic acid group, a hydroxyl group, an alkene, an alkyne, a ketone, an azido, or a primary amine group, or any combination thereof. The second portion may be selected based on available and/or reactive groups or moieties, such as amine or carboxyl groups, of the binding reagent. Y may be an alkyl or hydrocarbyl chain, having a sufficient number of carbon atoms to provide spatial or steric separation between the first portion and the second portion of the molecule. For example, the alkyl chain of the linkage portion may be long enough, e.g., with 5-20 carbon atoms, so that the molecules of the binding reagent have sufficient space to bind to the second portion of the molecule. The linkage portion may be an oligomer of any suitable monomer, such as an acrylate, styrene, or olefin monomer, and may be prepared using a controlled polymerization method for low polydispersity, such as controlled radical polymerization methods, e.g., atom-transfer radical polymerization.

“Alkyl” refers to straight, branched chain, or cyclic hydrocarbon groups, or combinations thereof, including from 1 to about 20 carbon atoms, for example and without limitation C₁₋₃, C₁₋₆, C₁_10, C₁-C₁₂, C₁₋₁₈ groups, for example and without limitation, straight, branched chain alkyl groups such as methyl, ethyl, propyl, butyl, pentyl, hexyl, heptyl, octyl, nonyl, decyl, undecyl, dodecyl, and the like.

Suitable linker molecules having a first portion, a second portion, and a linking portion that may be used include, but are not limited to, glutaraldehyde, (3-aminopropyl)triethoxysilane (APTES), L-Cysteine, thioglycolic acid, poly(ethylene glycol), N-hydroxysuccinimide esters, 11-mercaptoundecanoic acid, 12-mercaptodeodecanoic acid, or any combination thereof. Either of the first or second portion of the linking molecule may comprise the reaction product of two or more additional compounds. For example, as shown in FIG. 22, the carboxyl group of an 11-mercaptoundecanoic acid residue may be further modified with EDC-NHS to link to an amine group of a peptide, such as a protein.

For example, the protuberance may be a gold protuberance with 11-mercaptoundecanoic acid deposited thereover. The thiol group of the 11-mercaptoundecanoic acid molecule binds to the surface of the protuberance and the carboxylic acid group is available to react with the reactive groups or moieties of the binding reagent (FIG. 22). The protuberance may be a silver protuberance with a layer of gold deposited thereof, where the deposition of 11-mercaptoundecanoic acid reacts with the layer of gold as described above. The second portion of the molecule having a first portion, a second portion, and a linking portion is further reacted with a binding reagent, such that the binding reagent is covalently bonded to the surface of the protuberances. The binding reagent includes a reactive functional group or moiety (e.g., a primary amine) that is capable of reacting and covalently binding with the second portion (e.g., a carboxylic acid) of the molecule having a first portion, second portion, and linking portion deposited over the surface of the protuberances. The binding reagent may be bonded to the surface of the protuberance using be achieved using any suitable linking chemistry, such as by using carbodiimide chemistry. For example, the binding reagent may be covalently bonded to the protuberance by reacting the carboxylic acid second portion with a primary amine group present on the binding reagent to form an amide bond.

The binding reagent may covalently react with an electrically active material deposited over the surface of the protuberances, such that the binding reagent is covalently bonded to the surface of the protuberances (FIG. 2C). The reactive functional group or moiety (e.g., a primary amine) of the binding reagent is capable of reacting and covalently binding with the electrically active material deposited over the surface of the protuberances. The binding reagent can be coupled to the surface of the electrically active material-coated protuberance using any suitable linking chemistry, such as those described above. For example, the protuberances may be coated with a layer of graphene flakes, which may bind to the surface of the protuberances as reduced graphene oxide (rGO). In one example, carbodiimide chemistry, e.g., with EDC-NHS, can be used to link a reactive functional group of the binding reagent (e.g., a primary amine) to a carboxyl group of the rGO, to form an amide bond.

The term “binding reagent” refers to a compound having a binding moiety that binds specifically and non-covalently to a binding moiety of another compound, such as a specific target or analyte, referred to as its binding partner. The binding reagent and its binding partner bind non-covalently to form a binding pair. The strength of binding between members of a binding pair may be referred to as avidity. Non-limiting examples of binding pairs, include, but are not limited to: antigen/antibody, epitope/paratope (antigen binding site, ABS), lectin/carbohydrates, complementary nucleic acids, or aptamer/aptamer target.

The binding reagent on the 3D electrode array described herein may be a protein, an antibody, an antibody fragment, an epitope-containing polypeptide, an antigen, an aptamer, a nucleic acid, an affimer, or a combination of any of the preceding. The binding reagent may be an antigen or epitope of a protein of a coronavirus, such as SARS-CoV-2, ebola virus, human immunodeficiency virus (HIV), influenza virus, a herpes virus, zika virus or a bacteria, such as E. coli or M. tuberculosis. For example, the binding reagent may be SARS-CoV-2 spike S1 protein or SARS-CoV-2 spike receptor binding domain (RBD) protein. The binding reagent may be an antiviral antibody or antibody fragment, such as an antibody targeting an antigen of a coronavirus such as SARS-CoV-2, an ebola virus, a human immunodeficiency virus (HIV), influenza virus, a herpes virus or a zika virus. For example, the binding reagent may be an anti-SARS-CoV-2 spike S1 antibody or an anti-SARS-CoV-2 spike RBD antibody.

The binding reagent may comprise an antigen. The term “antigen” refers to a compound, composition, or substance that is produced by pathogen that stimulates an immune response in an animal, including, without limitation, proteins, polysaccharides, and glycoproteins. An antigen may be an oligopeptide or polypeptide comprising one or more epitopes of a protein of a pathogen. For example, the antigen may be a protein or an enzyme that is present on the surface of the pathogen. As used herein, “pathogen” refers to an infectious agent or pathogen, such as a bacteria, fungus, parasite, or a virus. For example, the binding reagent may be an antigen of a bacteria, such as Escherichia coli or Mycobacterium tuberculosis. The binding reagent may be an antigen of a virus, such as a coronavirus such as SARS-CoV-2, an influenza virus, a herpes virus, a human immunodeficiency (HIV) virus, an ebola virus, or a zika virus. Examples of suitable antigens include proteins of a pathogen that commonly elicits antibodies, such as neutralizing antibodies, including, without limitation: a coronavirus spike protein, an influenza virus hemagglutinin or neuraminidase protein, an ebola virus glycoprotein, or a zika virus envelope protein, as are broadly-known. The entire protein, or antigenic portions or fragments of an antigenic protein may be used in the structures, devices, and methods described herein. For example, the S1 portion of SARS-CoV-2, or E80 ectodomain of the zika envelope protein may be used. Useful antigens, such as fragments of larger proteins may be identified by their ability to specifically bind (for example) antibodies generated by a patient. Antigenic proteins are broadly-known in the art and typically are described in public databases, such as GenBank and UniProt. For example, SARS-CoV-2 proteins are provided in NCBI Reference Sequence: NC_045512.2, UniProtKB—PODTC2 (SPIKE_SARS2), and UniProtKB—PODTC9 (NCAP_SARS2, nucleocapsid), and fragments of the spike protein, e.g., S1 and S2 also may be used in the devices and methods provided herein. Determining appropriate antigens for use in binding antibodies found in patient's blood is well within the abilities of those of ordinary skill in the relevant arts.

The binding reagent may be an antibody or an antibody fragment, including engineered antibody fragments. The term “antibody” refers to an immunoglobulin, derivatives thereof which maintain specific binding ability, and proteins having a binding domain which is homologous or largely homologous to an immunoglobulin binding domain. As such, the antibody operates as a ligand for its cognate antigen, which can be virtually any molecule. Natural antibodies comprise two heavy chains and two light chains and are bi-valent. The interaction between the variable regions of heavy and light chain forms a binding site capable of specifically binding an antigen (e.g., a paratope). The term “VH” refers to a heavy chain variable region of an antibody. The term “VL” refers to a light chain variable region of an antibody. Antibodies may be derived from natural sources, or partly or wholly synthetically produced. Many antibodies and fragments thereof are available from commercial sources. An antibody may be monoclonal or polyclonal. The antibody may be a member of any immunoglobulin class, including any of the human classes: IgG, IgM, IgA, IgD, and IgE.

The term “antibody fragment” refers to any derivative of an antibody which is less than full-length. In exemplary embodiments, the antibody fragment retains at least a significant portion of the full-length antibody's specific binding ability. Examples of antibody fragments, but are not limited to, Fab, Fab′, F(ab′)₂, Fv, Fd, dsFv, scFv, diabody, triabody, tetrabody, di-scFv (dimeric single-chain variable fragment), bi-specific T-cell engager (BiTE), single-domain antibody (sdAb), or antibody binding domain fragments. The antibody fragment may be produced by any means. For instance, the antibody fragment may be enzymatically or chemically produced by fragmentation of an intact antibody, or it may be recombinantly or synthetically produced. The antibody fragment may optionally be a single chain antibody fragment. Alternatively, the fragment may comprise multiple chains which are linked together, for instance, by disulfide linkages. The fragment may also optionally be a multi-molecular complex. A functional antibody fragment may consist of at least about 50 amino acids or at least about 200 amino acids. Antibody fragments also include miniaturized antibodies or other engineered binding reagents, such as scFvs, that exploit the modular nature of antibody structure, comprising, often as a single chain, one or more antigen-binding or epitope-binding (e.g., paratope) sequences and, at a minimum, any other amino acid sequences needed to ensure appropriate specificity, delivery, and stability of the composition (see, e.g., Nelson, A L, “Antibody Fragments Hope and Hype” (2010) MAbs 2(1):77-83).

As used herein, the term “epitope” refers to a physical structure or moiety on a molecule that interacts with an antibody or antibody fragment.

“Lectins” are a group of proteins from non-immune origins that bind carbohydrates and agglutinate animal cells. They exhibit extremely high binding affinities for specific sugars, and can be used to target specific cell types expressing their binding partner or analytes, including carbohydrates, polysaccharides, glycoproteins, and glycolipids. Lectins can agglutinate cells and/or precipitate complex carbohydrates and, as such, have served as a powerful tool for biomedical research and clinical utility, including, carbohydrate studies, fractionation of cells and other particles, lymphocyte subpopulation studies, mitogenic stimulation, blood group typing, and histochemical studies. They are isolated from a wide variety of natural sources, both plant and animal Concanavalin A (Con A) is a broadly-studied lectin that binds α-D-mannosyl and α-D-glucosyl residues. Peanut agglutinin targets Galβ1-3GalNAcα1-Ser/Thr and, e.g., inhibits T-cell activity and can be used to distinguish lymphocyte subsets. Many other lectins are broadly-known and characterized, and can be obtained from commercial sources.

“Aptamers” are oligonucleotides or peptides that are selected to bind specifically to a desired molecular structure. Peptide aptamers are also referred to as “affimers”. Aptamers typically are the products of an affinity selection process similar to the affinity selection of phage display (also known as in vitro molecular evolution). The process involves performing several tandem iterations of affinity separation, e.g., using a solid support to which the desired immunogen is bound, followed by polymerase chain reaction (PCR) to amplify nucleic acids that bound to the immunogens. Each round of affinity separation thus enriches the nucleic acid population for molecules that successfully bind the desired immunogen. In this manner, a random pool of nucleic acids may be “educated” to yield aptamers that specifically bind to aptamer target binding partners. Nucleic acid aptamers typically are RNA, but may be DNA or analogs or derivatives thereof, such as, without limitation, peptide nucleic acids and phosphorothioate nucleic acids.

The binding reagent may be a nucleic acid or an analog thereof, e.g., a single-stranded nucleic acid or analog thereof, for binding complementary nucleic acid sequences found in a sample. “Complementary” refers to the ability of a single-stranded nucleic acid to hybridize to another single-stranded nucleic acid under assay conditions by Watson-Crick, or Watson-Crick-like base pairing of complementary bases. As such, free nucleic acids in a biological sample may be detected using the described electrode assembly where a binding partner of the free nucleic acid, that is, a complementary nucleic acid, is linked to the electrode array as described herein.

Also provided herein is a microfluidic test device. FIG. 1 depicts schematically a simplified version of a microfluidic device and a method of making the device. The microfluidic test device (e.g., as in FIG. 1 (f)) includes a microfluidic channel that may be prepared using PDMS, or another suitable composition, and which comprises an inlet for dispensing liquid into the microfluidic channel; an outlet for liquid to exit from the microfluidic channel. The microfluidic test device also includes an electrode assembly comprising a suitable substrate (e.g., glass as depicted in FIG. 1). The electrode assembly also comprises electrical traces, such as gold traces, for example as depicted in FIG. 1 (a), including a reference electrode (RE), a counter electrode (CE), and a working electrode (WE). At least the working electrode (WE) includes protuberances (see, FIG. 1 (c)), deposited by aerosol jet printing for directly contacting fluid in the microfluidic channel.

The substrate of the electrode assembly may be glass, silicon, silicon dioxide, plastic, or any combination thereof. The substrate may be patterned with a layer of a conductor, such as chromium, gold, silver, platinum, carbon, nickel, or indium tin oxide to form the electrode traces for the reference electrode, the counter electrode, and the working electrode. The electrode traces may be deposited in any useful pattern.

To form the reference electrode traces on the substrate, a conductive material may be coated onto the base of the reference electrode to form the reference electrode by methods known in the art, such as electrodeposition, dip coating, spray coating, curtain coating, or doctor (or draw-down) blade coating. A shadow mask may be used to apply the reference electrode material onto the substrate. The reference electrode material may be an ink containing silver/silver chloride (Ag/AgCl), gold, chromium, platinum, carbon, nickel, indium tin oxide, or any combination thereof. The reference electrode may also be a Calomel electrode, a pseudo silver electrode, or a combination thereof.

The counter electrode may be formed on the substrate in the same manner as the reference electrode and may comprise any suitable conductive material, e.g., as described above. A suitable material may be coated onto the counter electrode trace.

The 3D working electrode array is deposited onto the conductive working electrode trace. As with the reference and counter electrodes, the trace of the working electrode may be any suitable conductor, such as, without limitation: gold, chromium, platinum, silver, copper, or PEDOT. The 3D electrode array is AJ printed directly onto the working electrode trace. Alternatively, the 3D electrode array may be printed on a separate substrate and the substrate having the 3D electrode array may be adhered to the base of the working electrode using an electrically-conductive adhesive, such as an electrically-conductive glue, paste, or tape.

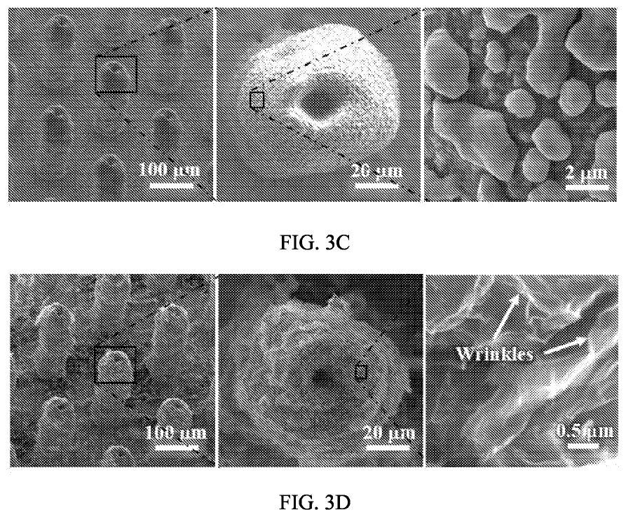

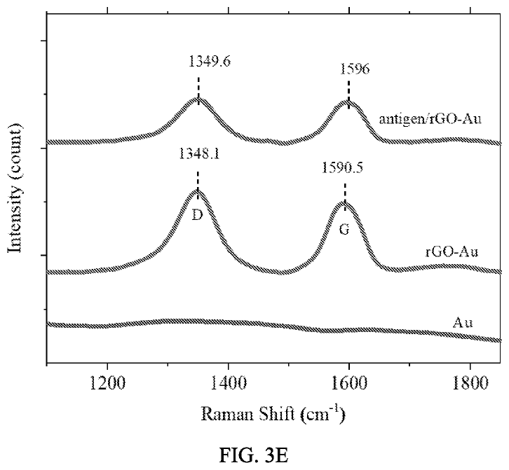

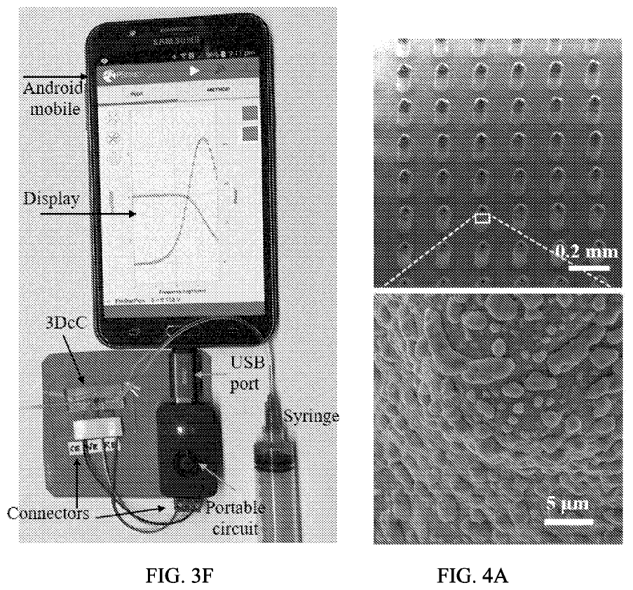

The portion of the working electrode having the 3D electrode array is in direct contact with the microfluidic channel (FIG. 1 (f), FIGS. 3A-3B). The remaining portion of the working electrode, that is, the trace, is not in direct contact with the microfluidic channel (FIG. 1 (f)). At least a portion of the reference electrode and at least a portion of the counter electrode are in direct contact with the microfluidic channel (FIG. 1 (f)). The remaining portions, that is, the traces, of the reference electrode and the counter electrode are not in direct contact with the microfluidic channel (FIG. 1 (f)).

Alternatively, droplet-based printing may be used to deposit the conductive material in a two-dimensional, but not necessarily flat pattern over or in contact with a conductive trace or lead on a substrate, without protuberances. The conductive material may be sintered as described above, resulting in a uniquely roughened surface amenable to modification. As above, for the protuberances, the surface may be then coated with another electrically-conductive material as described above, such as a reduced graphene oxide. Then the surface of the electrode may be functionalized with any binding reagent as described above. A benefit to this two-dimensional electrode is that it may be useful in detection of analyte, as described herein, where the sensitivity and high density functionality of the protuberances may be unnecessary or may be contraindicated. Also, a mixed electrode surface comprising two-dimensional portions and portions comprising protuberances may be provided, by which means sensitivity of the electrode may be tailored for the binding reagent/analyte combination.

The electrode may also include additional leads, or wires, attached independently to the reference electrode, the counter electrode, and the working electrode. The leads are then connected to a controller system. The module may send electrical signals to the leads. The module may receive an electrical signal from the electrode assembly. The controller system includes any required elements for powering the system, such as batteries, e.g., rechargeable batteries and/or a power supply, such as a DC power supply for powering other local components of the system, signal amplifier(s), microprocessors, non-transient memory such as read-only memory, hard drives, or flash drives, random access memory, analog-to-digital (A/D) converters, and/or communication modules for communicating with other parts of the system, either wirelessly, or wired. The module may include a controller, memory, a communications module, an input component, and an output component. It is understood that all or part of the controller system may be a separate, wired structure, or integrated into the substrate. The controller system may comprise a BIOS, one or more signal amplifiers, one or more analog-to-digital converters, memory, storage, processor(s), additional electronic hardware components, and/or computer-readable instructions for controlling the module and/or for communication within the system or with a separate device, for example, as are known in the computing arts. The controller system may be contained within a single housing or distributed among two or more devices.

An external module having a communication module for communicating wirelessly or by a wired connection may be connected to the controller system. The external module may include one or more computers, storage, and a communications module for communicating with other elements and, optionally, additional computers and/or computer networks. The external module also may comprise a BIOS, one or more signal amplifiers, one or more analog-to-digital (A/D) converters, memory, storage, processor(s), additional electronic hardware components, and/or computer-readable instructions for controlling the module and/or for communication within the system or with a separate device, e.g., as are known in the computing arts. The external module may be a computer, such as a personal computer, a laptop, a smartphone, or a dedicated controller device. The external module may communicate with the module via a wireless connection, such as by near-field communication (NFC), Zigbee, or Bluetooth protocols. The external module may be connected to any suitable output device, including displays, printers, or may communicate with one or more additional external devices, such as computers or computer networks via any suitable communication means, such as a computer network or over the Internet.

The controller system may comprise a controller for executing functions related to receipt, analysis, and transmission of sensed electrical data. For example, the controller may be a central processing engine including a baseline processor, memory, and communications capabilities. The controller can be any suitable processor comprising computer readable memory and configured to execute instructions either stored on the memory or received from other sources. Computer-readable memory can be, for example, a disk drive, a solid-state drive, an optical drive, a tape drive, flash memory (e.g., a non-volatile computer storage chip), cartridge drive, and control elements for loading new software.

The controller may include an executable program, code, set of instructions, or some combination thereof, executable by the controller system for independently or collectively instructing the controller system to interact and operate as programmed, referred to herein as “programming instructions”. In some examples, the controller is configured to issue instructions to initiate data collection from the electrode assembly and to select types of measurement information that should be recorded. In other instances, the electrode assembly may comprise suitable electronics and instructions configured to automatically transmit electrical signals to the module either in real time or at periodic intervals without first receiving initiation instructions from the controller system to initiate sensing and data transmission.

Processing can include applying filters and other techniques, hardware and/or software based, for removing signal artifacts, noise, baseline waveforms, or other items from captured signals to improve readability. Processing information includes data analysis techniques, such as quantifying various electrical signals based on received data, corroborating or calibrating data from multiple sources, and/or analyzing generated electrical signals to draw conclusions.

The controller system may further comprise an input component and an output component in communication with the controller, which allow a user to interact with and receive feedback from the module. The input component may include one or more of a keyword, touchpad, computer mouse, trackball, or other data entry accessory, for example as are known in the art. The input component can be used to enter information about the test sample which can be used to analyze the measurement data and/or to assist in analysis and training regimens. The input components can also be used to interact with a user interface by, for example, being able to toggle through instruction screens for configuring the electrical signals provided to or received from the sensing electrode. User interface screens that can be shown on a visual display and used for entering information and guiding a user in collecting information about a sample.

The components of the electrode assembly, controller system, and external modules can be combined in various manners with various analog and digital circuitry, including controllers, filters, ADCs (analog-digital chips), memory, communication devices and/or adaptors. As devices become smaller and processors become more powerful and use less energy, it is possible to integrate many more electrical and electronic components into the module. Technologies such as package on package (PoP) and system on a chip (SoC) integrated circuit packages allow manufacture of very small devices with significant capacities. For example, smart phones use PoP technologies to stack memory and processors in a very small volume. One example of a SoC is a microcontroller (MCU), which is a small computer on a single integrated circuit typically containing a processor core, memory, and programmable input/output peripherals. MCUs also may include timer module(s) and analog-to-digital converter(s) for, e.g., converting analog sensor output to a digital signal.

The microfluidic test device includes a microfluidic portion that includes a microfluidic channel, an inlet flowing into the microfluidic channel, and an outlet flowing out of the microfluidic channel. The microfluidic portion of the microfluidic test device may be fabricated using a soft-replica molding method (see, e.g., FIG. 1 (e)) or through other methods known in the art. The microfluidic portion of the test device may comprise a polymer. As used herein, “polymers” can include without limitation, homopolymers, heteropolymers, co-polymers, block polymers, block co-polymers, and can be both natural and synthetic. Homopolymers contain one type of building block, or monomer, whereas co-polymers contain more than one type of monomer. An “oligomer” can be a polymer that comprises a small number of monomers, such as, for example, from 3 to 100 monomer residues. As such, the term “polymer” can include oligomers.

The microfluidic portion of the microfluidic test device may comprise polydimethylsiloxane (PDMS), polymethyl methacrylate (PMMA), glass, sapphire, or a combination thereof. For example, the microfluidic portion of the microfluidic test device may be PDMS, where the microfluidic portion of the microfluidic test device is formed using a soft-replica molding method.

The microfluidic channel may be less than or equal to 5 mm in depth, such as less than or equal to 2 mm in depth, such as less than or equal to 1 mm in depth, such as 0.5 mm in depth. The width of the microfluidic channel may vary from 2 mm to 0.5 mm. For example, one part of the microfluidic channel may be 2 mm wide, and another part of the microfluidic channel may be 1 mm wide.

The inlet delivers a fluid to be analyzed into the microfluidic channel (FIG. 1 (f)). The inlet may be tubing, such as TYGON® tubing. In examples, one end of the inlet is connected to the microfluidic channel and the opposite end of the inlet is connected to a fluid delivery device, such as, for example and without limitation, a medical syringe or pump, which can be used to deliver the fluid into the inlet (see, e.g., FIG. 3F). The inlet may also be used to remove the fluid from the microfluidic channel.

The fluid may be a biological fluid, such whole blood, serum, plasma, urine, or saliva. The fluid may contain an analyte that is the binding partner of the binding reagent that is coated on the 3D electrode array. The fluid may not contain an analyte that is the binding partner of the binding reagent that is coated on the 3D electrode array.

For example, the 3D electrode array may be coated with an antigen or epitope of a protein of a coronavirus, an ebola virus, a human immunodeficiency virus (HIV), an influenza virus, a herpes virus or a zika virus and the fluid to be analyzed may have an antibody of a coronavirus, an ebola virus, a human immunodeficiency virus (HIV), an influenza virus, herpes virus, or a zika virus. The 3D electrode array may be coated with an antibody of a coronavirus, an ebola virus, a human immunodeficiency virus (HIV), an influenza virus, a herpes virus or a zika virus and the fluid to be analyzed may have an antigen or epitope of a protein of a coronavirus, an ebola virus, a human immunodeficiency virus (HIV), an influenza virus, a herpes virus or a zika virus. The 3D electrode array may be coated with an antigen or epitope of a protein of a bacteria, such as E. coli or M. tuberculosis and the fluid to be analyzed may have an antibody of a bacteria, such as E. coli or M. tuberculosis. The 3D electrode array may be coated with an antibody of a bacteria, such as E. coli or M. tuberculosis, and the fluid to be analyzed may have an antigen or epitope of a protein of a bacteria, such as E. coli or M. tuberculosis. The 3D electrode array may be coated with a spike S1 protein of SARS-CoV-2 and the fluid to be analyzed may be plasma collected from an individual with COVID-19 or suspected as having COVID-19. The 3D electrode array may be coated with a spike RBD protein of SARS-CoV-2 and the fluid to be analyzed may be plasma collected from an individual with COVID-19 or suspected as having COVID-19. The 3D electrode array may be coated with a spike S1 antibody of SARS-CoV-2 and the fluid to be analyzed may be plasma collected from an individual with COVID-19 or suspected as having COVID-19. The 3D electrode array may be coated with a spike RBD antibody of SARS-CoV-2 and the fluid to be analyzed may be plasma collected from an individual with COVID-19 or suspected as having COVID-19.

The microfluidic test device may also include a plasma separator, which is a microfluidic channel that can separate plasma from red blood cells. The plasma separator reduces pre-processing requirements by allowing the direct use of blood samples, e.g., heparin-treated blood samples, in the microfluidic test device. The plasma separator may include a single channel that splits into two separate channels, where the split forms a Y-shape. According to the Zweifach-Fung bifurication law, a channel bifurcates into two outlets, forming a Y-shape junction, with a minimum flow ratio of 2.5:1, separates blood cells from the plasma due to the variation of fluidic pressure at the Y-shaped junction. The plasma may then be directed to the microfluidic channel in direct contact with the sensing electrode for analysis and the blood cells may be discarded through an outlet.

The fluid that enters the microfluidic channel directly contacts the reference electrode, the counter electrode, and the working electrode. A fluid that contains the binding partner to the binding reagent that is coated on the 3D electrode array is sensed or detected by the 3D electrode array on the working electrode. The binding of the binding partner to the binding reagent to form a binding pair may be sensed or detected based on chemical impedance spectroscopy, cyclic voltammetry, chronoamperometry, or differential pulse voltammetry. If the fluid does not contain the binding partner to the binding reagent a signal will not be sensed or detected. Different amounts of the binding partner analyte to the binding reagent in the sample to be tested may result in different signals, permitting calibration and quantification of the analyte.

The outlet of the microfluidic test device permits egress of fluid, such as excess fluid, from the microfluidic channel. Excess fluid includes any fluid that was not analyzed or had been previously analyzed. Fluid can be channeled through the microfluidic channel from the inlet to the outlet to permit analysis of volumes of fluid greater than the volume of the microfluidic channel. For example, fixed volumes of fluid, such as 1, 2, 5, or 10 mL of fluid, can be passed through the microfluidic channel depending on expected analyte concentration, and calibration methods for the device.

The binding partner analyte may be eluted from the binding pair after analysis, thereby regenerating the binding reagent on the 3D electrode array. The sensing electrode may be rinsed with an acidic aqueous solution having a pH that is greater than 2.0 and less than 7.4. For example, the acid may be formic acid having a pH of 2.5. Other liquids, such as aqueous solutions and/or non-aqueous solutions may be used to elute analyte from the electrode assembly, and may be combined for optimal elution and preservation of the electrode assembly. Elution solutions may include solutions with different, e.g., non-physiological, salt concentrations, alcohols, surfactants, emulsifiers, etc. The 3D electrode array may further be rinsed with, for example and without limitation, phosphate buffered saline (PBS) or normal saline after completing an elution step, such as an acid rinse. As such, the electrode assembly can be used to analyze a fluid more than once, such as two times, such as three times, such as four times, such as five times, such as six times, such as seven times, such as eight times, or such as nine times, or more.

The microfluidic test device may include two or more sensing electrodes in direct contact with the microfluidic channel, where each of the sensing electrodes are the same or are different. The microfluidic test device may include a single sensing electrode which may include two or more working electrodes, where each of the working electrodes are the same or different. When different, the two sensing electrodes or two working electrodes may be used to test for at least two different analytes simultaneously, for example two different coronavirus antigens, or antigens from two different variants of a virus (such as two SARS-CoV-2 variants), or two different viruses or other infectious diseases simultaneously. The microfluidic test device may include two, three, four, or more different (multiplex) sensing electrodes or two, three, four, or more working electrodes, for example to detect two or more different infectious diseases. For example, the microfluidic test device may include a sensing electrode or a working electrode specific for the detection of a coronavirus, such as SARS-CoV-2, and a sensing electrode or working electrode specific for the detection of influenza virus.

The sensing electrode having the 3D electrode array may alternatively be open to the air without being in contact with a microfluidic channel, for example within a chamber configured to house the electrodes and having an opening into which a liquid may be deposited to contact the electrodes. For example, a fluid to be analyzed may be applied to the one or more 3D electrode arrays of the sensing electrode(s) as a droplet.

The following examples are presented to demonstrate the general principles of the invention. The invention should not be considered as limited to the specific examples presented.

### Example 1

### Materials and Methods

To construct the 3D array electrode using aerosol jet (AJ) printing, a commercial gold (Au) nanoparticle ink (UTDAu40, UT Dots Inc., Champaign, Ill.) was used. The average Au particle size was 4 nm, the ink viscosity was 3 centipoise (cP), and particle loading in the ink was 40 weight percent (wt %). The Au nanoparticles were dispersed in an organic non-polar solvent, which was aerosolized during AJ printing via ultrasound energy. Polydimethylsiloxane (PDMS) (SYLGARD™ 184 Silicone Elastomer Kit, Dow Corning, Midland, Mich., USA) with a base to hardener ratio of 10:1 was used to create the microfluidic channel of the 3D Printed COVID-19 Test Chip (3DcC) device.

Human recombinant SARS-CoV-2 spike S1-His protein (50 micrograms per milliliter (μg/mL)) and SARS-CoV-2 spike RBD-His protein (50 μg/mL) expressed in HEK293 cells, were the antigens purchased from the Sino Biological US Inc., Wayne, Pa. Before immobilizing on the 3D electrode surface, both the antigens were diluted to 5 μg/mL using a carbonate buffer solution having a pH of approximately 9.6. Two rabbit IgG antibodies, SARS-CoV-2 spike S1 antibody (10 microliters (μL)), and SARS-CoV-2 spike RBD antibody (10 μL) were also obtained from Sino Biological US Inc., Wayne, Pa. Both the antibodies were diluted in phosphate buffer saline solution (pH 7.4) containing a 5 millimolar (mM) ferro/ferricyanide before their introduction into the microfluidic channel for measurement. These solutions were stored at −20 degrees Celsius (° C.) before their use. Mouse monoclonal antibody (MAb) of human recombinant SARS-CoV-2 nucleoprotein (Cat. No. 40143-MM05) was purchased from Sino Biological Inc., Wayne, Pa. E. coli derived human recombinant interleukin-6 (IL-6; Cat. No., 206-IL) antigen was purchased from R&D Systems, Inc., Minneapolis, Minn. Bovine serum albumin (BSA), sodium bicarbonate, sodium carbonate, formic acid, phosphate buffered saline (PBS) powder, EDC (1-ethyl-3-(3-dimethylaminopropyl) carbodiimide hydrochloride) and NHS (N-hydroxysuccinimide) were acquired from Sigma Aldrich, St. Louis, Mo. A room temperature curable silver/graphene conductive epoxy (type G6E-RTSG, Graphene Supermarket, Inc., Ronkonkoma, N.Y.) was used to connect wires to the pads of the working electrode (WE), counter electrode (CE), and reference electrode (RE) (FIG. 1) of the 3DcC device.

Fabrication of Electrodes for 3DcC Device: FIG. 1 shows the fabrication process of the 3DcC device for rapid detection of COVID-19 antibodies. First, a patterned Au layer (with a chromium (Cr) adhesion layer) was deposited on a glass slide (FIG. 1 (a)) which formed the base for the three electrodes, namely, RE, WE, and CE. To pattern the Au/Cr layer, a shadow mask was created using a Kapton tape which was cut using an automated cutter (Silhouette Curio™, Silhouette America®, Inc., Lindon, Utah) with the aid of AutoCAD software (AutoCAD 2015, Autodesk Inc., San Rafael, Calif.). A 5 nm thick Cr layer was deposited as an adhesive layer, followed by a 100 nm thick Au layer using an e-beam evaporator (Kurt Lesker PVD 75, Jefferson Hills, Pa.) while using the Kapton shadow mask. The area of the patterned WE was 2 mm×2 mm Aerosol Jet nanoparticle 3D printing was then used to fabricate the three-dimensional micropillar electrode on the WE as shown in FIG. 1 (b-d).

The schematic of the AJ 3D printer (Model AJ-300, Optomec, Inc., Albuquerque, N. Mex. USA) is shown in FIG. 1 (b). The AJ printer consisted of an ultrasound atomizer, a deposition head with a nozzle, a movable (X-Y direction) platen with temperature control, and a shutter to break the flow of the aerosol as necessary. The ultrasonic atomizer created a mist of aerosol droplets of about 1-5 μm diameter from the Au ink, with each droplet containing the Au particles. A carrier gas (nitrogen, N₂) transported the droplets to the nozzle of the deposition head, while a sheath gas (also N₂) helped focus the nanoparticle beam to a length scale of 10 μm. The carrier gas pressure was set to be about 24 sccm and sheath gas pressure to set to 60 sccm. The nozzle diameter used for printing was 150 μm. Before printing, the geometry of the micropillar array was drawn in AutoCAD using a program in the software AutoLISP (AutoCAD 2015, Autodesk Inc., San Rafael, Calif.) and converted to a “prg” file compatible with the AJ printer software. One milliliter gold nanoparticle ink without any dilution was loaded inside a glass vial (FIG. 1 (b)), atomized using ultrasonic energy, and transported to the AJ printer. An external heating element was placed on top of the X-Y stage to heat the substrate to 150° C. A layer-by-layer printing sequence was used to build up the 3D 10×10 micropillar arrays from AJ droplets where the path of the droplets could be periodically blocked (or cleared) by controlling the shutter. The schematic of complete micropillar array is shown in FIG. 1 (c). The printing of an individual pillar is shown in FIG. 1 (d), where a layer of droplets was deposited on top of the previously printed and solidified Au micropillars. The strong surface tension of the printed ink caused the pillar to form without any support structures (FIG. 1 (d)). Once a layer is printed, the ink loses solvents due to the heat from the platen. The dried ink provides a base to receive the next micro-ring; and the process is repeated. The printing process for the 10×10 micropillar array was completed within 35 minutes for a given printhead. After printing, the dried micropillar array was heated to 400° C. for 5 hours to completely remove the binders and sinter the Au nanoparticles which completed the manufacture of the WE of the device. The surface of the RE on the glass substrate (FIG. 1 (a)) was then coated with commercial silver/silver chloride ink (Ercon, Inc. Wareham, Mass.) by using a Kapton shadow mask. The curing temperature for this coat was 150° C. for 2 hours. The ink composition comprised of dispersed chloridised silver flake particulates in a solvent. Compared to thin film Ag/AgCl reference electrode, the commercial ink was chosen due to its improved stability as demonstrated in literature (Ali, M. A. et al. Continuous Monitoring of Soil Nitrite Using a Miniature Sensor with Poly(3-octyl-thiophene) and Molybdenum Disulfide Nanocomposite, ACS Appl. Mater. Interf 2019, 11, 29195).

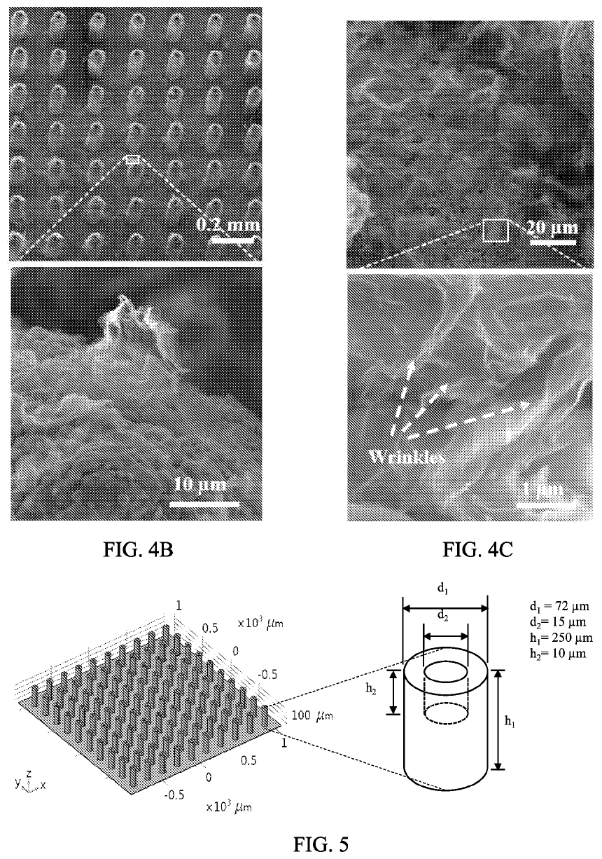

The process of the functionalization of the WE is shown in FIGS. 2A-2C. First, the gold micropillars (FIG. 2A) were functionalized with reduced graphene oxide (rGO) (FIG. 2B). The rGO nanoflakes (i.e., sheets) were obtained in powder form (CAS-No. 7782-42-5, ACS Materials LLC, Pasadena, Calif.). The rGO sheets were dispersed in deionized (DI) water (0.2 milligrams per milliliter (mg/mL)) and sonicated for 2 hours. As per the manufacturer, the rGO was obtained from graphene using a reduction process based on hydrazine (N₂H₄) treatment. Per the manufacturer data sheet, the rGO sheets had a conductivity >500 Siemens per meter (S/m), a diameter 0.5-10 μm, and a thickness of approximately 1 nanometer (nm). Before coating, a PDMS fence was created and placed surrounding the Au micropillar array. A 20 μL rGO solution was drop-cast onto the Au micropillar array using a pipette and dried at 80° C. for an hour. This process was repeated three times. The Au pillars were covered by rGO sheets as observed by scanning electron microscopy (SEM) images (FIGS. 3D, 4B, and 4C) and Raman analysis (FIG. 3E). The Au micropillar-rGO surface was further functionalized using SARS-CoV-2 antigens (FIG. 2C). This was achieved by using a coupling reagent consisting of a mixture of EDC (0.2 Molar (M)) and NHS (0.05 M) in a ratio of 1:1 by volume. A 20 μL solution of the EDC:NHS mixture was spread over the rGO-Au surface to activate the carboxylic acid (—COOH) groups of the rGO sheets. The electrode on the glass substrate was kept in a humid chamber (at approximately 100% of humidity) for four hours and washed with PBS solution. Next, a 20 μL of SARS-CoV-2 spike S1 antigen solution (5 μg/mL) was spread on the surface of the rGO-Au array electrode via drop-casting using a pipette (10-50 μL; Cole-Parmer; Vernon Hills, Ill. 60061) and kept for 4 hours in a humid chamber and then washed with PBS. The activation achieved by EDC:NHS chemistry enabled the primary amine (—NH₂) groups of the protein molecules (i.e., antigens) of SARS-CoV-2 spike to form C—N bonds with the —COOH groups of rGO sheets via an amidation reaction. In this reaction, the EDC is known to act as a cross-linker and NHS as an activator (Liu, E. Y. et al., High-throughput double emulsion-based microfluidic production of hydrogel microspheres with tunable chemical functionalities towards biomolecular conjugation, Lab Chip 2018, 18, 323). Another SARS-CoV-2 antigen called spike receptor-binding domain (RBD), was also immobilized on a different sensor using same mechanism as that described above. For the RBD antigen, a 20 μL solution (5 μg/mL) was used for drop-casting over the Au micropillar-rGO electrode. For all sensors, a 20 μL of BSA solution (2 mg/mL) was introduced to the WE surface to block any non-specific sites of the antigen conjugated rGO-Au pillar. In this device, the 3D electrodes acted as sensitive immuno-detectors for COVID-19 antibodies without any labelling agents via antibody-antigen interactions (FIG. 2D).

Fabrication of PDMS housing and Assembly of the 3DcC Device: The PDMS housing of the 3DcC device was fabricated by soft-replica molding method (FIG. 1 (e)) and integrated with the glass containing functional electrodes (FIG. 1 (f)). A thick polymethylmethacrylate (PMMA) mold was created using high-precision milling machine that had a channel with 1 millimeter (mm) depth, 2 mm and width, and 2 centimeter (cm) length. The channel width in the middle 1 cm section was increased to 2 mm (FIG. 1 (e), Step 1). The dimensions of sections A and A1 in FIG. 1 (e), step 1 are 1×1×5 cubic millimeters (mm³), and 2×1×10 mm³, respectively. This structure then acts as a mold for the PDMS housing that contains a cavity for microfluidic channel as shown. A PDMS solution was poured on the PMMA channel to copy an opposite pattern (FIG. 1 (e), Step2). The bubbles were removed from the liquid PDMS mixer by degassing for 1 hour in a vacuum chamber (10⁻⁴ Torr). Curing temperature was 80° C. for 2 hours. The PDMS substrate was peeled off from PMMA channel (FIG. 1 (e), Step 2) and acted a master mold for creating the final microfluidic channel. The surface of this PDMS mold was treated with silicone oil (Ease Release™ 205, Reynolds Advanced Materials, Macungie, Pa.). A new mixer solution of PDMS was then poured into the PDMS mold (FIG. 1 (e), Step 3) and then peeled off, resulting in the PDMS channel required for the 3DcC device (FIG. 1 (e), Step 4). Holes were then punched at two ends of the channels by a hollow needle and tygon tubes were inserted for fluid injection. Finally, the PDMS slab was manually placed on the glass substrate such that the CE, WE, and RE were under the channel. Wires were connected to the pads of the electrode outside the PDMS channel using a room temperature curable silver/graphene conductive epoxy to connect to a potentiostat. A syringe was used to inject the testing fluid into the sensor in about 2 seconds. The volume of the entire microfluidic channel used for this study was 30 cubic millimeters (mm³), requiring 30 μL of fluid with the antibodies.

Electrode Characterization: An electrochemical workstation with Zview software (VersaSTAT 3 Potentiostat Galvanostat, Princeton Applied Research, Oak Ridge, Tenn.) was used to record the electrochemical signals and analyze the impedance spectra. In addition, a smartphone-based reading platform was enabled, where the sensor was interfaced with an Android mobile phone using a portable device (Sensit Smart Device, PalmSens, Inc., Randhoeve 221, GA Houten, The Netherlands). The Sensit Smart is a microcontroller-based system having a 30.5 mm×18 mm×2.6 mm system-on-module (SOM) potentiostat. The potentiostat device could be directly connected to a smart phone via a USB-C interface. Further, a mobile app ‘PStouch’ was used to record the data. The Au micropillar electrodes were imaged by a scanning electron microscope (FEI Sirion SEM, Hillsboro, Oreg.). The elemental analysis was carried out using Energy Dispersive X-ray spectroscopy (EDX) in the same instrument as the SEM imaging. The Raman spectra were collected using the NT-MDT AFM/Raman (NT-MDT America, Tempe, Ariz.) that used a 532 nm green laser with 12 milliwatt (mW) laser power for excitation.

Electrochemical Simulations: The modeling and 3D simulation of the different electrode structures was conducted using finite element software, COMSOL Multiphysics® (Version 5.5, COMSOL Inc., Burlington, Mass.). This study was carried out to investigate the diffusion profiles for different geometries of the electrodes and their corresponding electrochemical currents which were generated due to an electrochemical reaction at the surface of the electrode. In this electroanalysis scheme, a redox species B was considered to be oxidized to form a product (A) by losing an electron (B↔A+e⁻). At the boundary, the product concentration was zero, but the bulk concentration of oxidative species was taken to be 1 mole per cubic meter (mol/m³) and uniform. In this study, Fick's second law of diffusion was utilized as the domain equation which is given as:

$$\frac{\partial {c}_{i}}{\partial t} = ∇ \cdot ( {D}_{i} ∇ {c}_{i} ) \qquad Equation 1$$

wherein the cᵢ=1 mol/m³. The diffusivities were taken for the 2D and 3D electrodes as obtained from the cyclic voltammograms at different scan rates, along with the Randles—Sevcik equation (Elgrishi, N. et al., A Practical Beginner's Guide to Cyclic Voltammetry, J. Chem. Edu. 2018, 95, 197).

Calculation of Limit-of-Detection for the 3DcC Device: The limit-of-detection (LoD) was calculated using a method described by Armbruster et al. (Armbruster, D. A. et al., Limit of blank, limit of detection, and limit of quantification, The Clin. Biochem. Rev. 2008, 29, S49). The calculation involved evaluating limit-of-blank (LoB), the limit of detection of the signal (Y_{LOD}), and the LoD in terms of concentration (Lavín, Á. et al. On the Determination of Uncertainty and Limit of Detection in Label-Free Biosensors, Sensors 2018, 18, 2038) using:

LoB=Mean of signal (blank sample)+1.645×(Standard deviation of blank sample) Equation 2

Y_{LoD}=LoB+1.645×(Standard deviation of target at low concentration) Equation 3

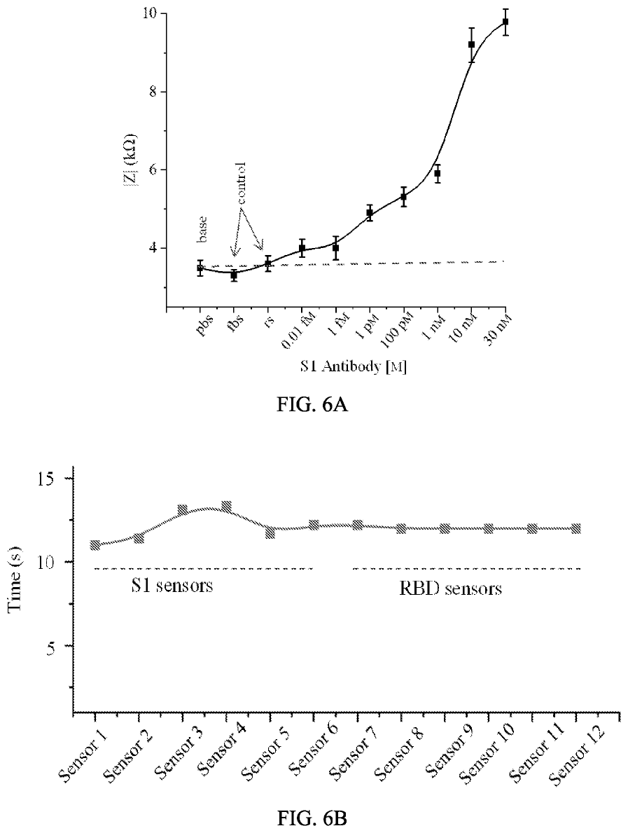

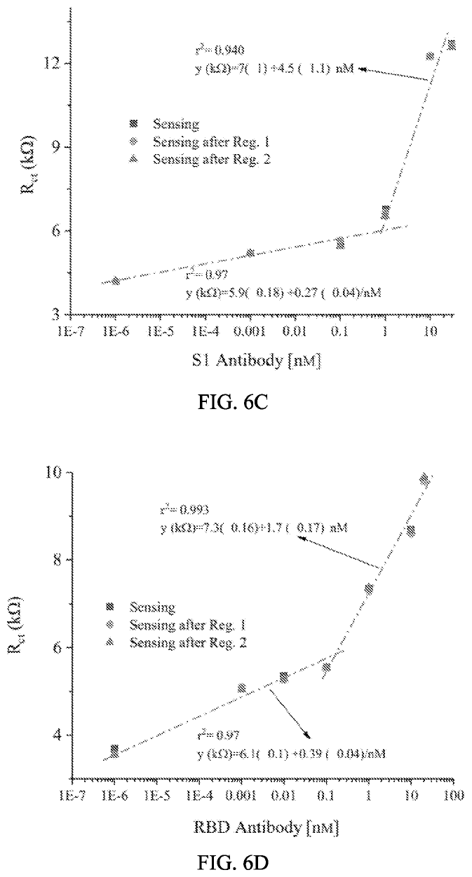

LoD=(Y_{LoD}−c)/(slope of the sensor calibration) Equation 4

where, ‘c’ is the intercept of the calibration curve of the sensor (FIG. 6C).

### Results

Design and Construction of COVID-19 Test Chip using Nanomaterials: The schematic of the 3DcC device along with AJ nanoprinting of the three-dimensional electrodes is shown in FIG. 1 (a-f). The functionalization of 3D electrodes by rGO nanoflakes and viral antigens is also a step in 3DcC device formation and is described in FIGS. 2A-2C. FIG. 1 (a) shows a glass slide coated with patterned chromium (5 nm thick) and gold (100 nm thick) which formed the base layer of the three electrodes (working electrode or WE, counter electrode or CE, and reference electrode or RE) of an electrochemical cell. FIG. 1 (b) shows the AJ printing process of the micropillar array on the WE shown in FIG. 1 (a). The AJ printer breaks the gold nanoparticle ink in the vial via ultrasonication into microdroplets, each containing the gold nanoparticles (2-5 nm in diameter). The microdroplets were carried to the nozzle via N₂ and aerodynamically focused on the WE, which was heated to 150° C. FIG. 1 (c, d) show a CAD (Computer Aided Design) controlled process of droplet dispense to form individual pillars. The deposition was in the form of micro-rings of gold nanoparticle ink. Once one layer was printed, the solvent evaporated due to the heat from the substrate, forming solidified ‘dry’ material containing the nanoparticles and binders. When the next ring was printed, the surface tension of the solvent in the ring allowed the micropillars to be built without the use of any support structures. Each layer of the rings was about 5-10 μm thick and formed within a fraction of a second. A succession of these processes formed a 10×10 micropillar array consisting of unsintered nanoparticles and binders. Upon sintering of the printed structure, the gold micropillars for the electrode were formed. FIG. 1 (e) shows the schematic of the construction of the PDMS housing by replica-molding method, which involved using polymethylmethacrylate (PMMA) mold and a polydimethylsiloxane (PDMS) mold to create a PDMS microfluidic channel. The RE, shown in FIG. 1 (a) was coated with a thin silver/silver chloride (Ag/AgCl) layer via a shadow mask. The PDMS housing was then placed manually on the glass slide containing the micropillar array electrode and other electrodes (CE and RE). The complete construction of the 3DcC device is shown in FIG. 1 (f). Note that fluid could be introduced in the device by tubes inserted into the microfluidic channel.

For detection of antibodies from the fluid introduced in the electrochemical cell, the 3D printed microelectrode was functionalized by viral antigens using rGO nanoflakes. This process is depicted in FIGS. 2A-2D. The bare 3D micropillar electrode is shown in FIG. 2A. The micropillar electrode array functionalized using rGO nanoflakes is shown in FIG. 2B. Electrostatic or Van der Waals interactions allow the rGO sheets to be connected to the micropillar. The surface porosity of the 3D printed micropillar shown in the SEM images in FIG. 3C likely aid in this process. The carboxylated (—COOH) groups of rGO are also shown in the FIG. 2B. Due to the π-π interactions amongst the coated rGO, the coating was expected to be non-uniform. FIG. 2C shows antigens bonded with the rGO nanoflakes of the electrode. This was achieved by activating the —COOH groups of rGO using a coupling chemistry of EDC:NHS. This chemistry facilitates the formation of C—N covalent bonding between rGO and antigens via an amidation reaction. Specifically, this reaction involved the EDC molecules acting as cross-linkers between —COOH and —NH₂ (i.e., amine group of the viral antigens) and NHS molecules acting as stabilizers during this reaction. Two SARS-CoV-2 viral antigens, namely, spike S1 and RBD, were separately used in this process (in different sensors). Other protein molecules could be attached using the same principle. Schematic in FIG. 2D shows antibodies selectively binding with the antigens when a fluid containing antibodies was introduced in the chamber. A treatment with BSA was used to block non-specific sites of the sensor, as described above.

The 3DcC device works on the principle of electrochemical transduction. When an AC potential is applied between WE and RE, an electrical double layer is formed at the WE-electrolyte interface (FIG. 2F). For an electrolyte such as ferro/ferricyanide, the double layer can be formed due to accumulation of oppositely charged ions at the interface compared to the charge on the electrode (FIG. 2F). This creates a characteristic impedance when a constant potential is applied between WE and RE. An equivalent circuit is shown in FIG. 2F, where Cal is the double layer capacitance, R_{ct} is the charge transfer resistance, and Rₛ is the electrolyte resistance. The electrolyte used was PBS solution mixed with ferro/ferricyanide. When a fluid containing antibodies to the antigens shown in FIG. 2D are introduced in the microfluidic chamber, the selective binding of the target antibodies to the corresponding antigens on the electrode was expected, which would increase the thickness of Cal layer, causing a higher R_{ct}, which could then be detected via electrochemical impedance spectroscopic (EIS) measurements (FIG. 2G). First, we expect the 3D geometry to accelerate the formation of the electrical double layer compared to a corresponding 2D surface, enabling a fast detection of the changes in R_{ct} and an increase in the sensitivity. Further, we expect that binding of the antibodies with antigens will increase the thickness of the double layer, causing the R_{ct} to increase proportionally. Thus, measurements of R_{ct} values provide a measure of the presence of antibodies in the fluid introduced in the 3DcC device.

Physical Characterization of 3DcC Device: FIGS. 3A-3D show the microscopic and spectroscopic analysis of 3DcC device constructed using the processes shown in FIGS. 1 and 2A-2D. An optical micrograph of the device with CE, WE, and RE is shown in FIG. 3A. The SEM images of 3D gold micropillar array electrode (i.e., WE) fabricated by AJ printing prior to rGO and antigen functionalization is shown in FIGS. 3B and 3C. The average and standard deviation of pillar height, pillar diameter, and pillar-to-pillar distance are 249.3±6.7 μm, 73.2±2.3 μm and 118.3±2.2 μm, respectively when measured at six random locations across five sensors.

The top surface of a solid pillar is pointed and had a 15 μm deep and a 20 μm diameter dip which was a result of the printing process depicted in the schematic of FIG. 1 (d). The surface texture of the gold pillars formed by nanoparticle sintering is shown in the zoomed-in SEM images of FIGS. 3C and 4A. This texture consisted of micron-sized gold crystals on the pillar surface and a surface porosity caused by the nanoparticle sintering process. This surface texture was expected provide additional sites for rGO adhesion, in addition to increasing the total surface area. The micropillar electrode after the rGO and antigen treatment (FIG. 2C) is shown in FIGS. 3D, 4B, and 4C. FIG. 3D shows the rGO nanoflakes attached to the gold micropillars, as well as the gold base layer (also see FIG. 4C). The rGO nanoflakes formed secondary three-dimensional networks adjacent to the micropillars. Graphene nanoflakes appeared wrinkled on the Au surface due to π-π interactions between graphene sheets (zoomed-in images of FIGS. 4B and 4C) which was expected to enhance the loading capacity of the antigens.

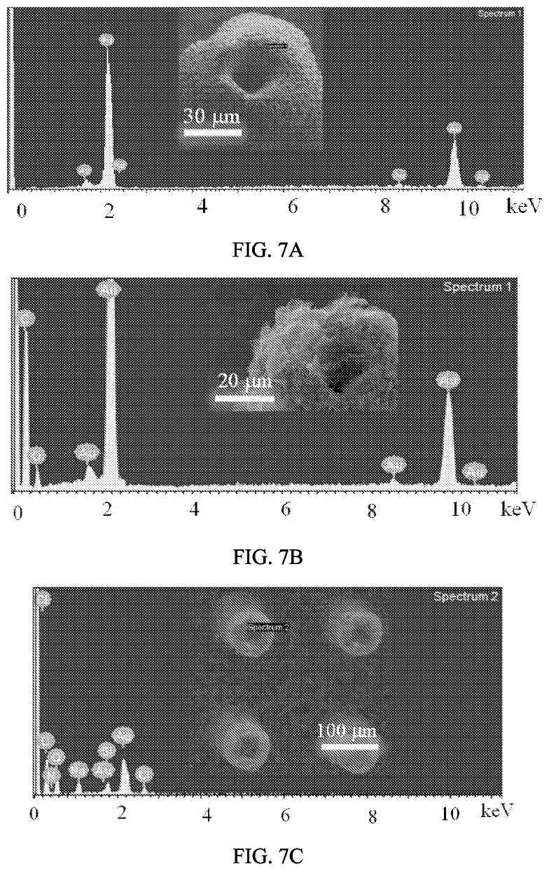

Raman spectroscopic measurements were conducted to investigate the defect (D) and graphitic (G) bands present in the rGO-Au micropillars (FIG. 3E) with and without antigens. The Au micropillar surface prior to rGO treatment did not show any Raman peaks. The Raman spectrum of rGO-Au micropillars revealed a D-band at 1348.1 inverse centimeters (cm-1) and a G-band at 1590.5 cm⁻¹. The D-band (symmetry A1g mode) is due to the vibration of −sp³ carbon atoms or defects while G-band originates due to first-order scattering of E2g phonon of sp² carbon atoms at the center of Brillouin zone. The appearing D and G peaks in the Raman spectrum indicate the presence of rGO sheets on gold pillars. The intensity ratio (I_{D}/I_{G}) of rGO-Au was 0.84, which was slightly changed to 0.85 after incorporation of antigens. Antigen immobilization on rGO sheets influenced the peak position, which was shifted to higher wavenumber (5.5 cm⁻¹) due to the decreased graphitic nature of the rGO sheets. EDX studies indicated presence of large amount of carbon when rGO was coated on the gold micropillars (Table 1 and FIG. 7B). The existence of nitrogen for antigen immobilized sample (Table 1 and FIG. 7C) may come from the —NH₂ group of the protein (i.e. antigen). The user interface for reading of the electrochemical cell signal was a laptop or a smartphone. A 3DcC device with a readout using a smartphone-based interface is shown in FIG. 3F.

TABLE 1
EDX results of micropillars before coating, after coating
with rGO, and after immobilization of antigens on rGO-Au

| | Au Atomic | | | |
|---|---|---|---|---|
| Electrodes | Percent (at. %) | C at. % | O at. % | N at. % |
| Au pillar | 100 | 0 | 0 | 0 |
| rGO-Au pillar | 6.7 | 84.8 | 8.4 | 0 |
| Antigen/rGO-pillar | 3 | 63.3 | 21 | 8.4 |

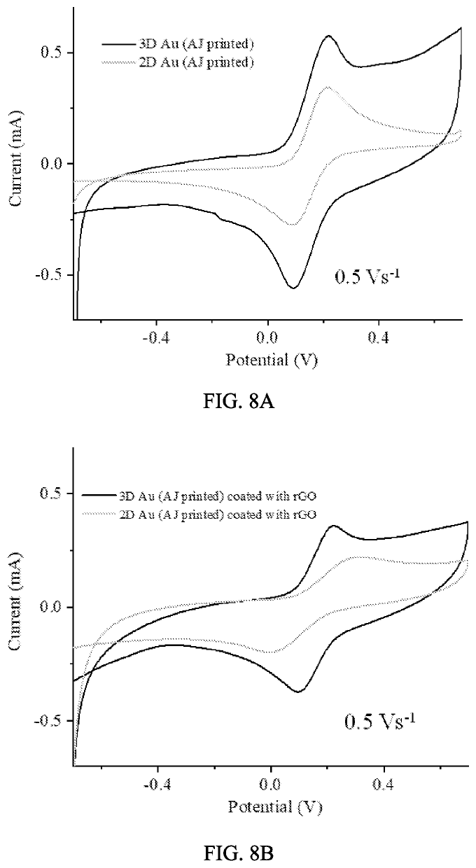

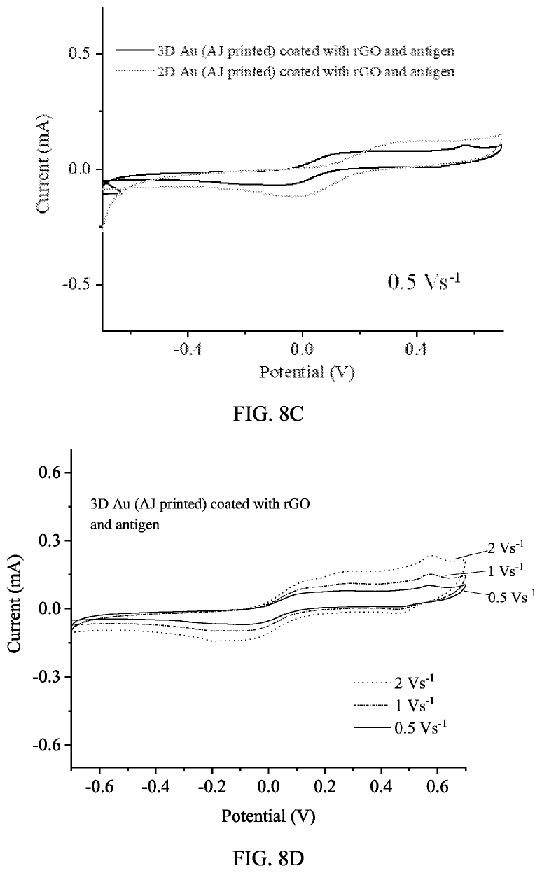

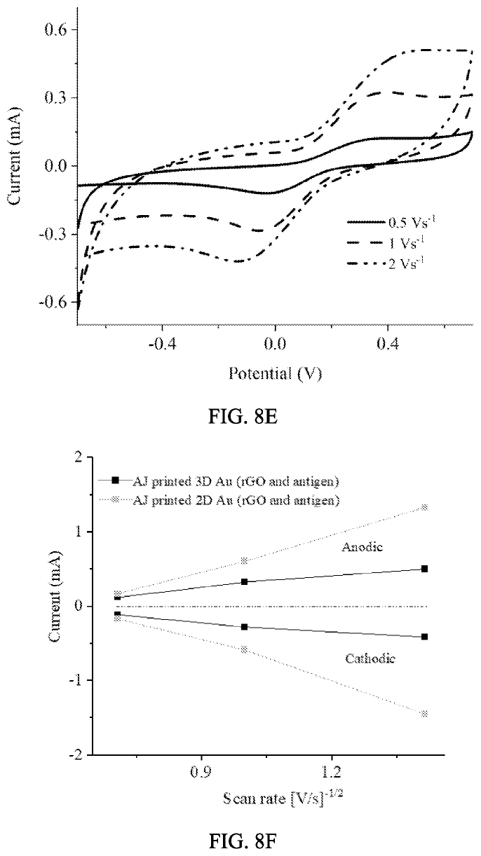

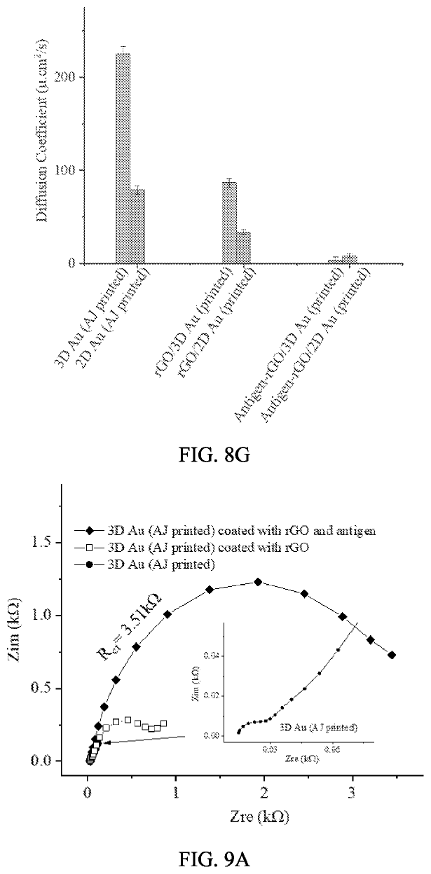

Electrochemical Characterization of 3DcC Device: Cyclic voltammetry (CV) studies were carried out to investigate the electrochemical properties of the AJ printed 3D Au electrode (i.e. with micropillar geometry shown in FIGS. 3B-3D) and compare it with AJ printed 2D Au electrode (FIGS. 8A-8G). The 3D Au (AJ printed) and 2D Au (AJ printed) electrodes showed clear oxidation and reduction peaks (FIG. 8A). The 3D Au electrode showed a 170% enhancement of current compared to the 2D Au electrode as shown in Table 2.

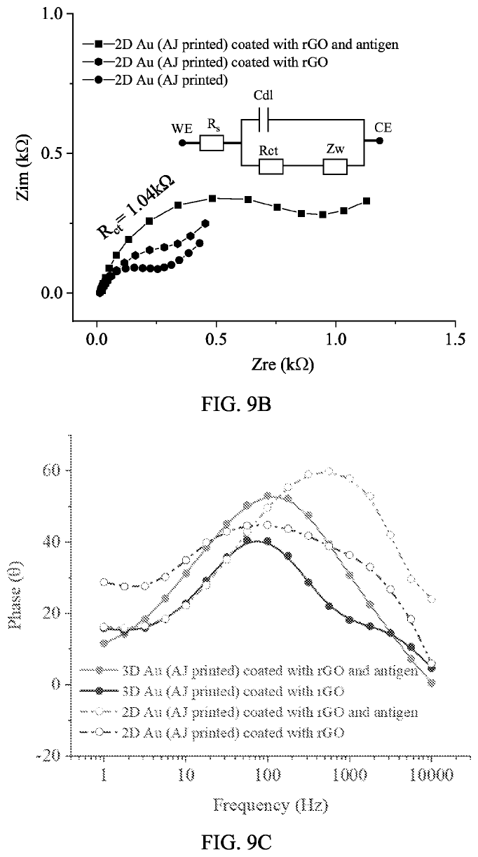

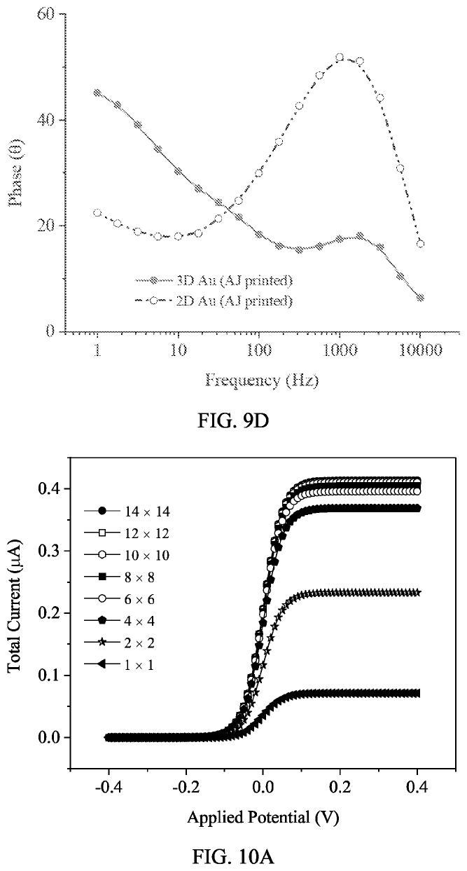

TABLE 2
The electrochemical parameters obtained from the data in FIGS. 8A-8G
and FIGS. 9A-9D for different electrodes (the standard deviation
represents four repeated measurements across two sensors).

| | | Peak-to-peak | | Charge |
|---|---|---|---|---|
| | Oxidation | potential | Diffusion Co- | transfer |
| | Current | difference | efficient | resistance |
| Electrodes | (μA) | (mV) | (μ · cm²/s) | (R_{ct}) (Ω) |
| 3D Au (AJ printed) | 571 ± 1.63 | 128 ± 2.9 | 225 ± 4 | 30 ± 0.2 |
| 2D Au (AJ printed) | 339 ± 7.3 | 121 ± 3 | 79 ± 2.5 | 390 ± 0.71 |
| 3D Au (AJ printed) | 354 ± 3.9 | 127 ± 1.1 | 86.8 ± 2.18 | 894 ± 1.2 |
| coated with rGO | | | | |
| 2D Au (AJ printed) | 221 ± 7.7 | 294 ± 3.2 | 33.8 ± 1.1 | 668 ± 7.1 |
| coated with rGO | | | | |
| 3D Au (AJ printed) | 75.4 ± 1.05 | 198 ± 2.2 | 3.9 ± 0.1 | 3510 ± 15.7 |
| coated with rGO | | | | |
| and antigen | | | | |
| 2D Au (AJ printed) | 110 ± 3.5 | 368 ± 5.4 | 8.3 ± 1.05 | 1040 ± 5.2 |
| coated with rGO | | | | |
| and antigen | | | | |

This is due to larger-surface area and porous feature of the AJ printed 3D Au electrode. The 3D Au electrode provided both radial and linear diffusion of electrons which enhanced the electrochemical current, while planar 2D Au electrode provided a linear diffusion of electrons. This was confirmed by COMSOL simulations. This difference is partly responsible for the high redox current of the 3D electrode. Further, the simulation shows that the diffusion of redox species reached equilibrium in 50s for electrodes with 2D geometry, while it took only 0.5s to reach equilibrium for the 3D micropillar geometry. The simulation indicates a higher concentration of redox species for the 3D micropillar electrodes when compared to 2D planar electrodes. After applying the rGO coating, the electrochemical signal was decreased (FIG. 8B) due to the presence of functional groups on the rGO surface that can hinder the electron transfer during the redox reaction. Further, the currents again decreased due to the immobilization of SARS-CoV-2 spike S1 antigens onto the two electrodes (FIG. 8C). To assess the mass transport of potassium ferro/ferricyanide, the scan rate studies were conducted (FIGS. 8D and 8E). The variation of anodic and cathodic peaks is plotted against the square root of scan rates (FIG. 8F) which indicates that these sensors showed a diffusion-controlled process. The diffusion coefficient of the 3D Au is 280% higher than the 2D Au without rGO and antigens; 256% higher than the 2D Au upon rGO coating; but reduced significantly upon loading of the antigen molecules. This indicates that the 3D geometry can hold higher amount of antigen molecules, thereby improving the sensor efficacy.

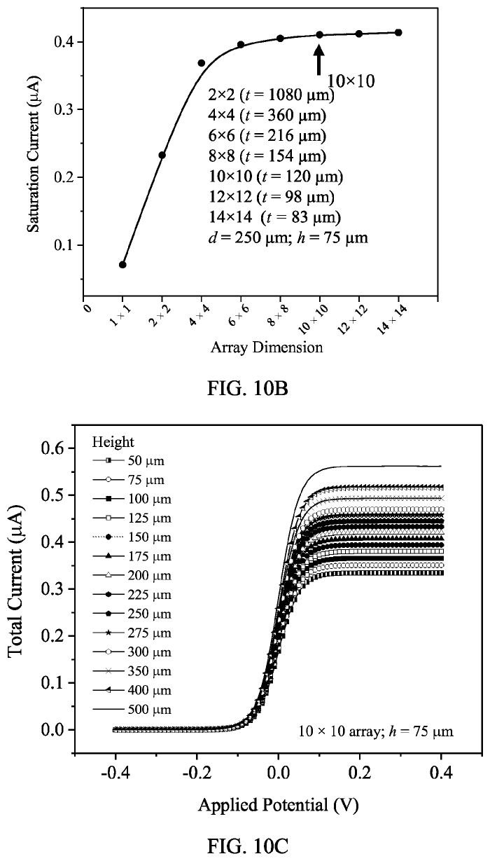

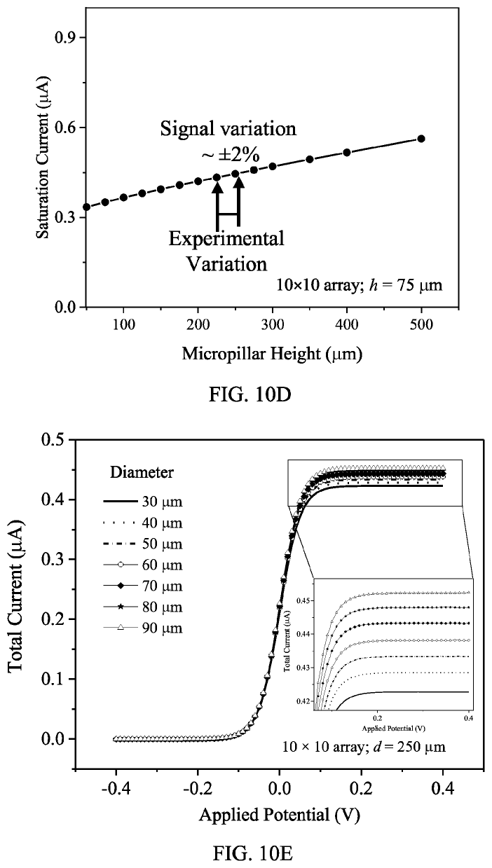

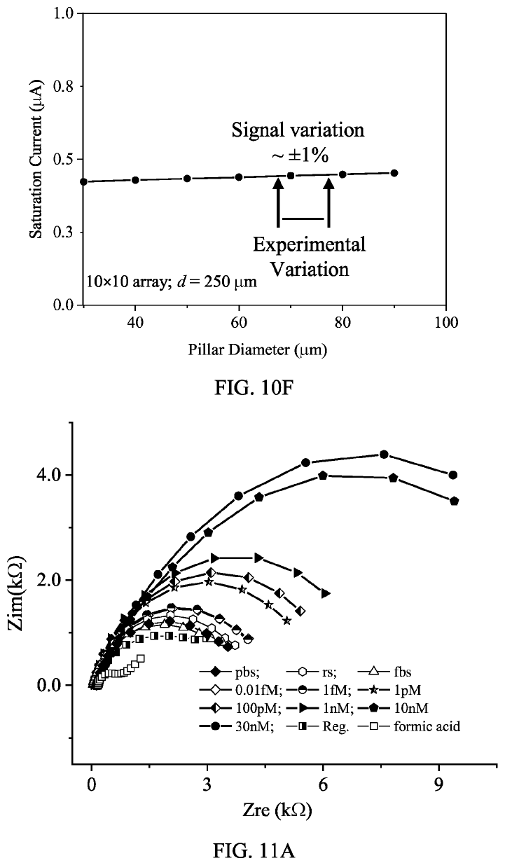

The electrodes were also characterized by the EIS method (FIGS. 9A-9D) to investigate the electrode-electrolyte interfacial properties. The R_{ct} value of the 3D Au electrode without any coating was 30Ω (FIG. 9A) possibly due to the highly conductive 3D microstructure and radial diffusion of the ions. While rGO sheets are incorporated onto the 3D micropillar, the R_{ct} is increased to 0.894 MI (Table 2) due to the presence of functional groups (COOH, OH) on rGO sheets. Further functionalization by SARS-CoV-2 spike S1 antigens on the 3D surface of rGO and Au increased the loading capacity of SARS-CoV-2 spike S1 antigen, leading to a maximum R_{ct} value of 3.51 kδ. However, with antigen and rGO coating, the AJ printed 3D Au electrode had the signal improvement of 330% compared to AJ printed 2D Au electrode. In phase shift (θ) plots (FIG. 9C), the printed electrodes showed higher θ values, while the electrodes are functionalized with spike S1 antigen. The θ value for AJ printed 3D Au electrode is found to be shifted to lower frequencies (100 Hz) compared to the AJ printed 2D electrode (FIG. 10C).

In order to understand the electrochemical processes in the 3DcC device, simulations of the CV experiments using COMSOL Multiphysics Software for the 2D and 3D electrodes were completed. The bulk concentration was assumed to be 1 mol/m³, while the diffusion coefficient was taken from that obtained in the experiments from the Randles-Sevcik equation. FIG. 5 shows the electrode geometry. The presence of radial diffusion as shown in COMSOL simulations confirms the speculation that this phenomenon may help create high redox current in case of the 3D electrodes.

COMSOL simulations were also conducted to investigate the effect of the AJ printed electrode geometry on the current of the electrochemical cell. The simulation results are given in FIGS. 10A-10F. The one-dimensional plots of currents versus applied potential are shown for various array configurations (FIG. 10A), pillar heights (FIG. 10C), and pillar diameters (FIG. 10E); while the corresponding currents are shown in FIGS. 10B, 10D, and 10F, respectively. The experimental range of values for the pillar dimensions is also indicated in FIGS. 10B, 10D, and 10F. The electrochemical current is saturated at the array size of 10×10 (FIGS. 10A and 10B). Further, the variations in the current for the experimental range of value is within ±2% for pillar height, ±1.0% for pillar diameter, ±3.2% for pillar-to-pillar distance.

Sensing of COVID-19 Antibodies: SARS-CoV-2 S and N proteins have been used for serologic assays for detecting antibodies of SARS-CoV-2 infection. The S protein binds to viral entry receptor angiotensin-converting enzyme-2 (ACE2) and mediates viral entry. It is present as a trimer with three receptor-binding S1 heads sitting on top of a trimeric membrane fusion S2 stalk. The receptor-binding subunit S1 contains the N-terminal domain (NTD) and the RBD while the membrane fusion subunit S2 contains the fusion peptide (FP), two heptad repeats (HR1 and 2), a transmembrane anchor (TM) and the intracellular tail (IC). Serologic assays using both recombinant S1 and RBD proteins have been shown to detect specific antibodies in COVID-19 patients. For antibody tests in this work, we thus chose the spike S1 and RBD antigens of SARS-CoV-2 to develop the 3DcC testing platform.

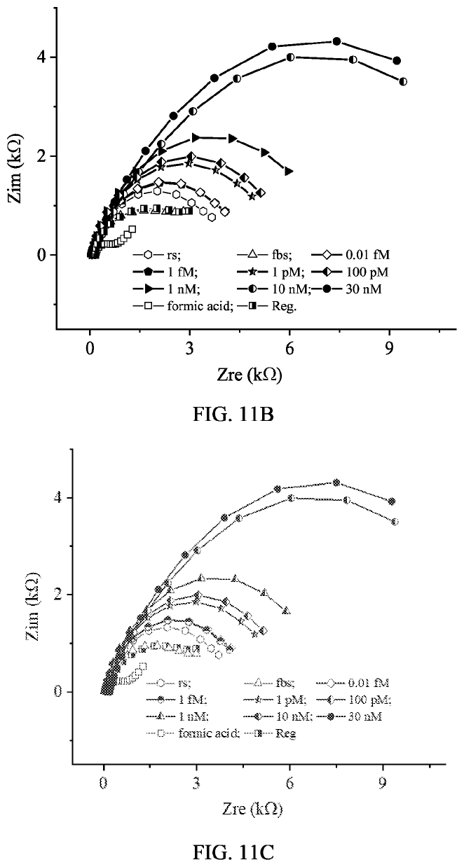

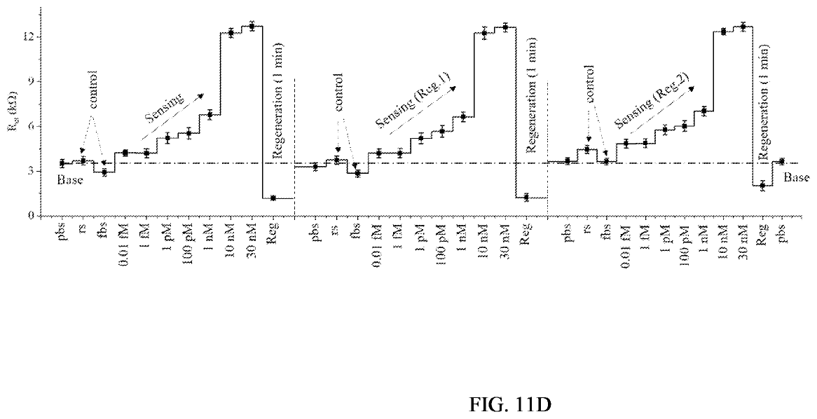

FIGS. 11A-11D shows the impedimetric sensing plots and R_{ct} for the 3DcC sensor when the electrodes are exposed to PBS, rabbit serum (rs), fetal bovine serum (fbs), and spike S1 antibodies (rabbit IgG). The spike S1 antibodies were introduced successively at concentrations of 0.01 fM, 1 fM, 1 pM, 100 pM, 1 nm, and 30 nM, three times each, wherein the sensor was regenerated after each set of measurements, including the controls testing. In the first set of measurements (FIG. 11A), a testing baseline was set by allowing only the PBS solution with no target antibodies which showed a charge transferred-limited process with R_{ct} of 3.51 kiloohms (kΩ). While the sensor was tested with biological samples such as rs and fbs as controls, the sensor generated similar profiles of Nyquist plots having a deviation of ±6.01% compared to the baseline signal. The adsorption of any proteins or albumins can change the sensor signals significantly. However, though these proteins have a major composition of albumin protein, the sensor did not show a significant change in the baseline signal. The sensor surface contained a layer of BSA molecules which are negatively charged, which may have repelled the albumin molecules in the serum due to their identical charge. This result indicated that the sensor was selective to specific proteins, as was desired. With these control measurements, the 3DcC device was tested with low concentrations of spike S1 antibodies at 0.01 fM and 1 fM. With the 0.01 fM concentration, a change of impedance signal (4.2 kΩ) was observed (FIG. 11D) compared to the sensor baseline and the control serum. This is as expected since binding of the antibodies with the antigens will obstruct electron transfer from the electrolyte to the electrode surface resulting in an increase in circuit impedance as captured by the Nyquist plot. Between 0.01 fM and 1 fM, the sensor did not show any significant change in impedance signal. Within this range of concentration of antibodies, the sensor surface may have only limited number of antibodies resulting in an unchanged signal. At 1 pM concentration of antibodies, however, the sensor showed a significant increase in the impedance to 5.21 kn. As the antibody concentration was increased to 1 nM, the R_{ct} increased further as reflected in the Nyquist plots. When a 10 nM concentration was tested, the sensor provided a very significant increase in the impedance signal (12.3 kΩ). Further, a minute change of signal (12.7 kΩ) was observed at 30 nM concentration. Testing beyond 10 nM concentration of spike S1 antibodies showed that the sensor exhibited a saturated impedance signal, wherein the maximum number of binding sites on the sensor surface are likely to be occupied by the target antibodies.

To elute antibodies from the sensor surface, we exposed the sensor electrodes to a solution of formic acid (1.0 M) having a pH of 2.5. This was chosen since a pH of 2.5 is outside the physiological range for human body (7-7.4 pH), where antibodies are expected to elute from the antigens via a disruption of immunoaffinity. Indeed, upon introduction of the formic acid solution for 60 seconds into the sensor, the R_{ct} values dropped to about 1.2 kn. In addition to eluting the antibodies, the low R_{ct} is caused by high electron transfer rates in acidic media. The signal from formic acid in FIGS. 11A-11C are after the 1^{st}, 2^{nd}, and 3^{rd} regeneration. After the 60 second incubation by formic acid solution, the sensor was washed with a PBS solution. At this point, the sensor recovered to 94% of its base signal. FIGS. 11A and 11C show sets of measurements after regeneration for the same set of spike S1 antibody concentrations as studied in FIG. 11A. After the 1^{st} regeneration, the signal with rb and fbs (FIG. 11B) was within ±6.96% of the baseline. After the 2^{nd} regeneration, this signal did not change significantly (FIG. 11C). We also observed similar patterns of impedance signals for spike S1 antibody solutions at different concentrations after the 2^{nd} regeneration as shown in FIGS. 11C and 11D.

For the S1 sensor, the mean of the blank signal and the standard deviation were 3499.4 Ohms (Ω) and 551Ω (n, replicate=5), respectively. From Equation 2, the LoB was 4406Ω. The mean signal at the lowest concentration (0.01 fM of S1 antibody) was 3481.1Ω, with a standard deviation of 16.36Ω [n=4]. The Y_{LoD} was 4433Ω, as calculated per Equation 3. The sensor calibration equation (FIG. 6C) was Y_{LoD} (kΩ)=0.27×Log [X (nM)+5.9]. This gave a c of 5.9 and a slope of the sensor calibration curve as 0.27. By plugging in these values into Equation 4, the LoD for the S1 sensor was determined to be 2.8 fM. The spike S1 sensor could provide a LoD and an analytical sensitivity of 2.8 fM, and 1 pM respectively.

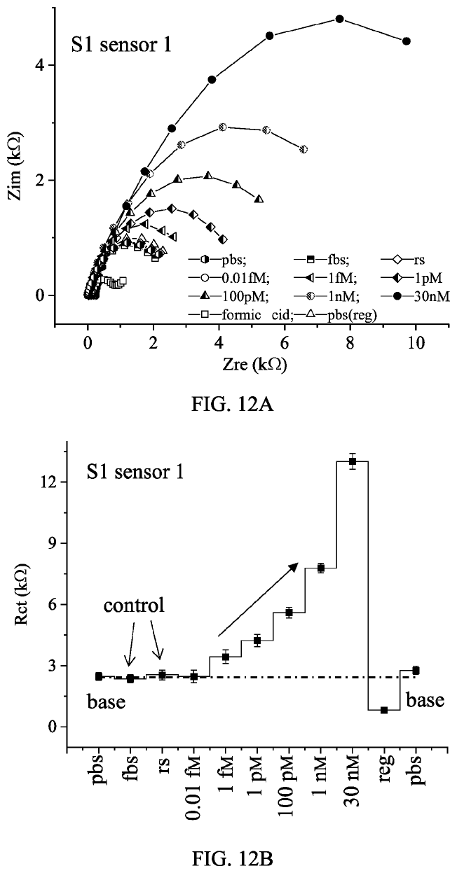

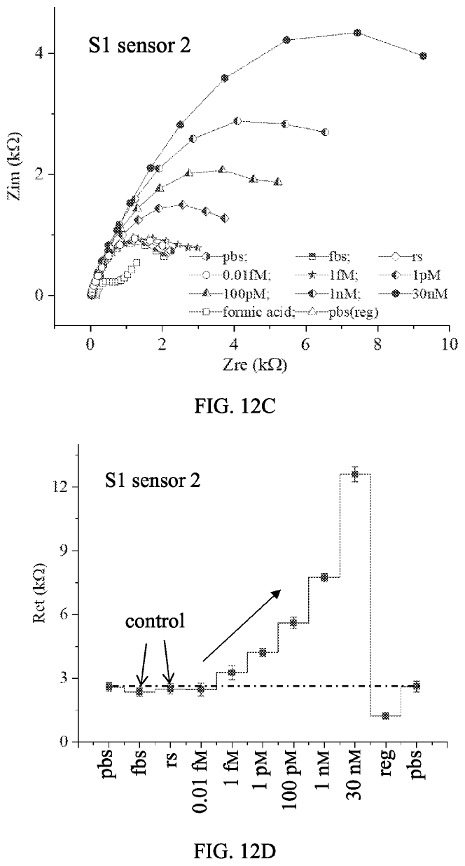

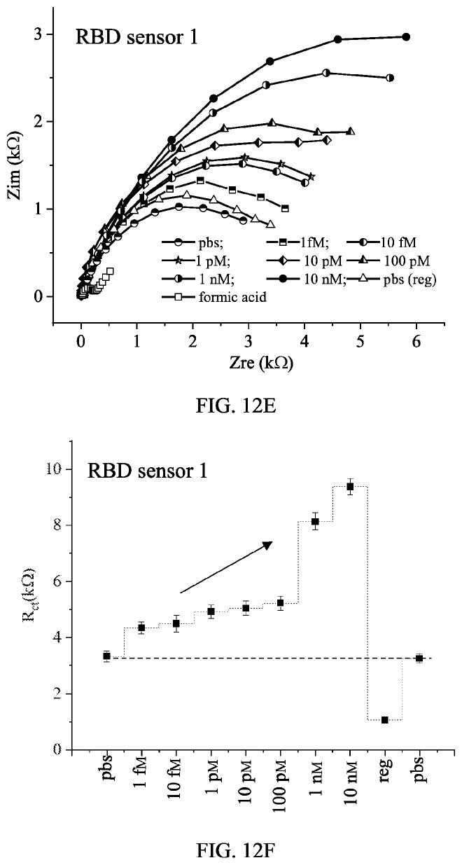

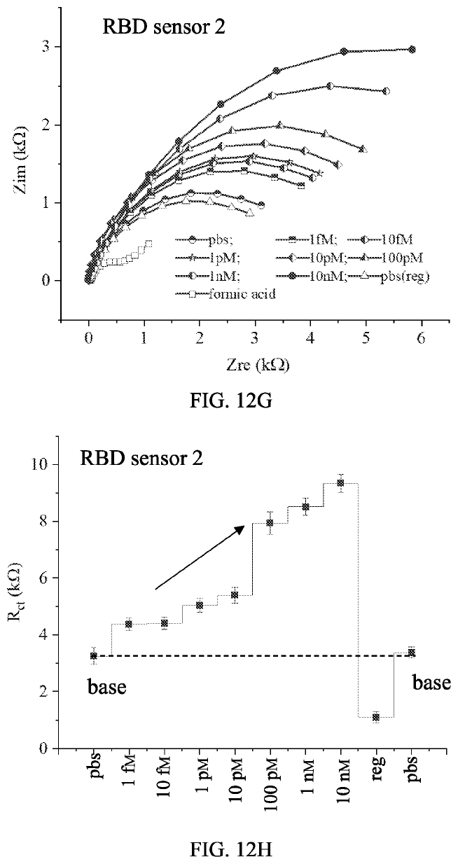

Two additional spike S1 sensors were also tested with serial dilution and results are shown in FIGS. 12A-12D.

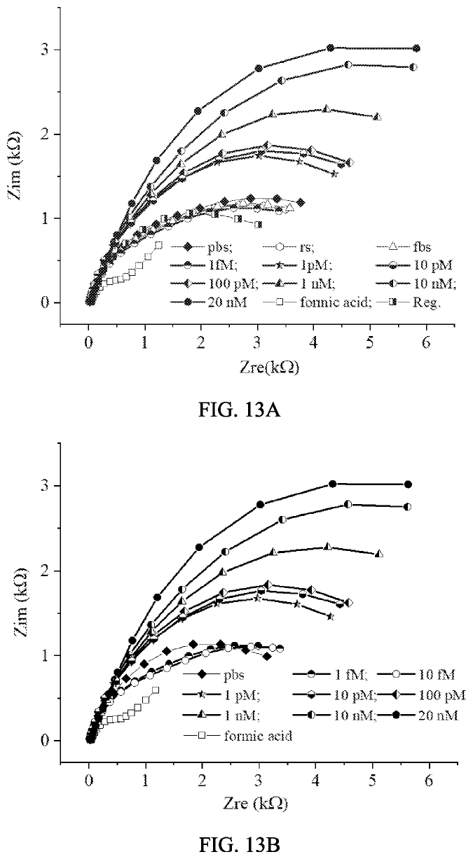

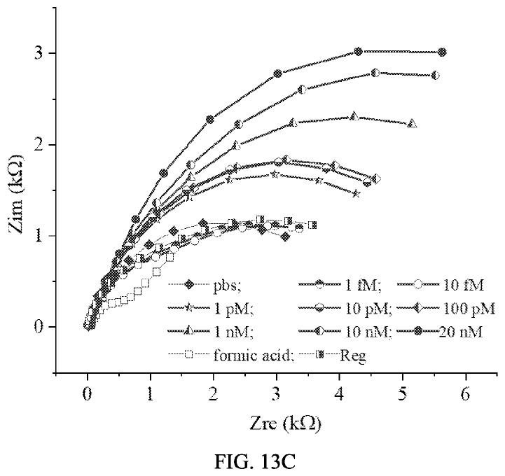

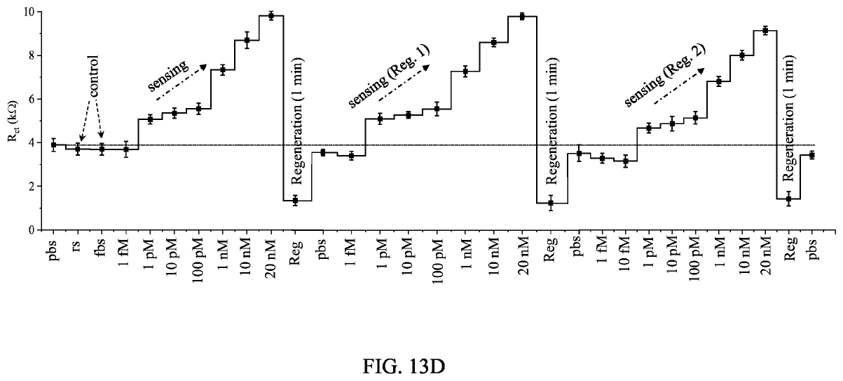

FIGS. 13A-13D shows the sensing of RBD antibodies using the 3DcC device where results are presented in a similar manner as that for spike S1 antibodies (FIGS. 11A-11D). The device had micropillar electrodes with immobilized SARS-CoV-2 RBD-His recombinant antigens. The EIS spectra for each concentration of RBD antibodies with control biofluids such as rs and fbs are shown in FIG. 13A; while that after two successive regenerations are shown in FIGS. 13B and 13C. The RBD antibody concentrations were set from 1 fM to 20 nM to test the sensor. The sensor base signal was obtained by collecting impedance spectra in presence of a PBS solution where the sensor showed a R_{ct} of 3.89 kΩ (FIG. 13D). With the rs and fbs, the sensor showed a deviation of 3.0% in the impedance after regeneration when compared to sensor baseline. The sensor did not show any increased signal when 1.0 fM concentration of RBD antibodies were introduced. At RBD antibody concentration of 1 pM, the R_{ct} value changed to 5.07 kn. Similar to the spike S1 antibodies, as the concentration of RBD antibodies was increased from 1 pM to 20 nM, the R_{ct} values were found to increase as shown in FIG. 13D. The regeneration of the RBD sensor was achieved by exposing the sensor to a 1.0 M formic acid (pH 2.5) solution for 60 seconds; the same as that used for the regeneration of spike S1 antibodies. Unlike the spike S1 sensor, the RBD sensor showed different linear responses between 1 pM to 100 pM, and 100 pM to 20 nM. After two regenerations, a minimum loss of sensitivity (±1%) was observed for the RBD sensor.

For the RBD sensor, the mean of the blank signal and standard deviation were 3781Ω and 75.99Ω (n, replicate=5), respectively. From Equation 2, this gave a LoB of 3781Ω. The mean signal at the lowest concentration (1 fM of RBD antibody) was 3422.6Ω, with a standard deviation of 201Ω for [n=4]. This gave a Y_{LoD} of 4236Ω, as calculated per Equation 3. The sensor calibration equation (FIG. 6D) was Y_{LoD} (kΩ)=0.39×Log [X (nM)+6.1]. This gave a c of 6.1 and a slope of the sensor calibration curve of 0.39. By plugging these values into Equation 4, the LoD for the RBD sensor was determined to be 16.9 fM. The RBD sensor could provide a low LoD and an analytical sensitivity of 16.9 fM, and 1 fM respectively.

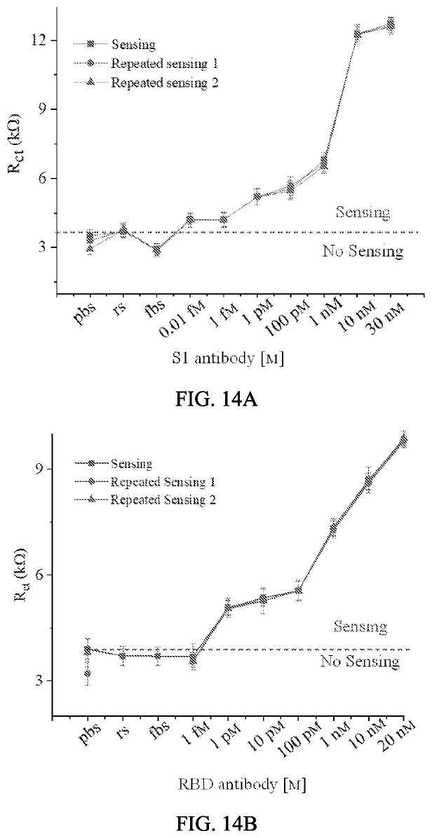

Further, sensing data for two additional sensors of spike RBD antibodies are shown in FIGS. 12A-12H. Based on the results in FIGS. 11A-11D and 13A-13D, we created a dose-dependent response plot for spike S1 and RBD sensing, respectively, where a minimum R_{ct} value was identified above which the sensing could be achieved (FIGS. 14A and 14B).

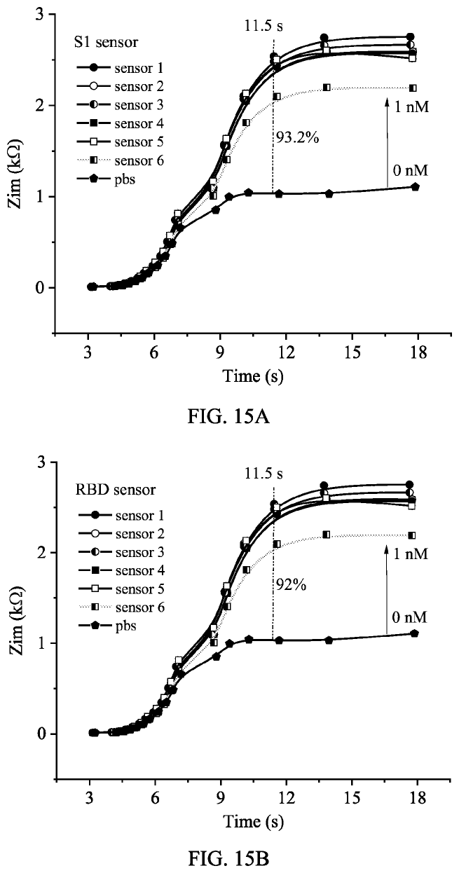

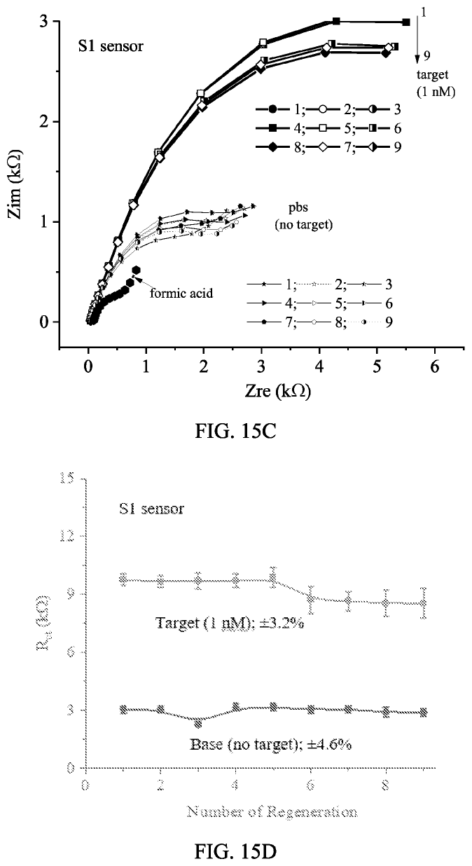

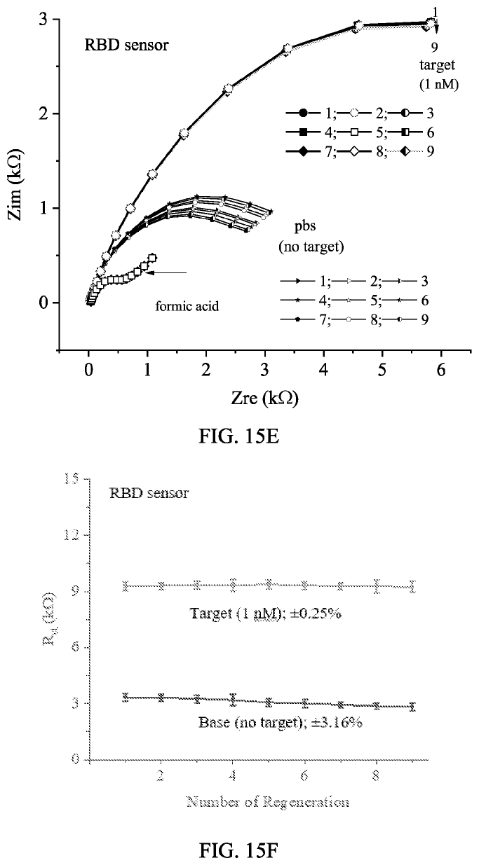

FIG. 15A shows the impedance of the sensor as a function of time (in seconds) for the detection of spike S1 antibodies. With no target in the PBS solution, the sensor impedance started to change at 3 seconds and reached approximately 1 kΩ in 10 seconds, beyond which it became saturated. When spike S1 antibodies (1 nM) were introduced in six different sensors, a significant change of impedance was observed (in line with data in FIGS. 11A-11D) and reached about 93.2% of the saturation value in 11.5 seconds, indicating detection of antibodies. The magnitude of impedance at 11.5 seconds as a function of the spike S1 antibody concentrations (0.01 fM to 30 nM) for the 3DcC device is shown in FIG. 6A. FIG. 15B shows the impedance as a function of time for six different 3DcC RBD sensors when RBD antibodies are introduced at 1 nM concentration. For all the sensors, the signal reached 92% of the saturation signal at 11.5 seconds, again indicating detection of the antibodies. FIG. 6B shows the time required to reach 95% of the saturation signal for the data presented in FIGS. 15A and 15B. Therefore, the 3DcC device can detect the antibodies to SARS-CoV-2 virus within seconds.

To validate the sensor regeneration phenomenon, 3DcC sensors for the detection of spike S1 antibody and RBD antibody were regenerated 9× each as shown in FIGS. 15C and 15D, and FIGS. 15E and 15F, respectively. A PBS solution was used as a reference while 1 nM antibody solutions were used as the target. After 9× regenerations, the spike S1 and RBD sensors did not show significant changes in the R_{ct} values as evidenced by their low relative standard deviation (RSD) of less than ±3.2% and ±0.25%, respectively. However, the base line R_{ct} of spike S1 and RBD sensors was found to change by ±4.6% and ±3.1% when compared to that with no regeneration. After 9 regenerations (i.e., 10th reading), both sensors provided a good signal, indicating a high regeneration capability. The acidic media may degrade the bonding between rGO and antigens after repeat exposures, possibly limiting the regeneration capability beyond a certain limit.

The sensitivities of the 3DcC device for spike S1 and RBD sensors are shown in FIGS. 6C and 6D. These plots are obtained from the R_{ct} values in FIGS. 11D and 13D. For spike S1 sensor, the slope of FIG. 6C was 0.27±0.04 kΩ/nm in the range of 1.0 fM to 1 nM, and 4.5±1.1 kΩ/nm in the range of 1 nM to 30 nM. The high sensitivity at higher concentrations is likely due to higher number of antibodies captured by the sensing surface. For RBD sensors the slope of FIG. 6D was 0.39±0.04 kΩ/nm for a range of 1.0 fM to 0.1 nM, and 1.7±0.17 kΩ/nm for a range of 0.1 nM to 20 nM. The spike S1 sensor showed a higher sensitivity at a higher concentration of antibodies when compared to the RBD sensor. This high sensitivity of the 3DcC device was likely due to the 3D architecture, a high porosity, and specific surface chemistry that allowed an enhanced loading capacity of the antigens.

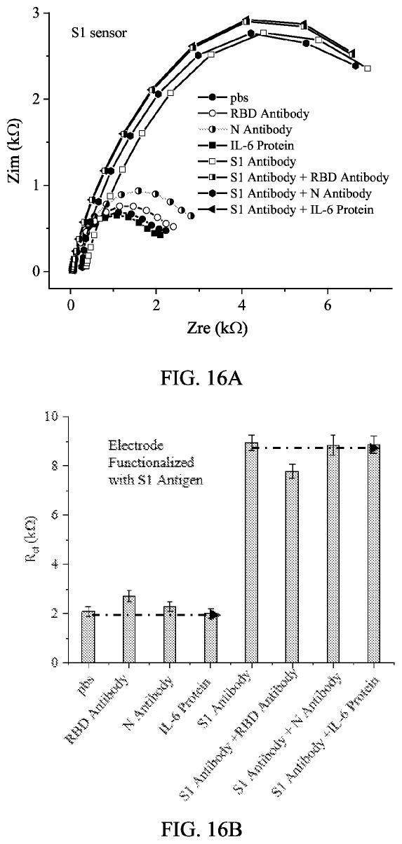

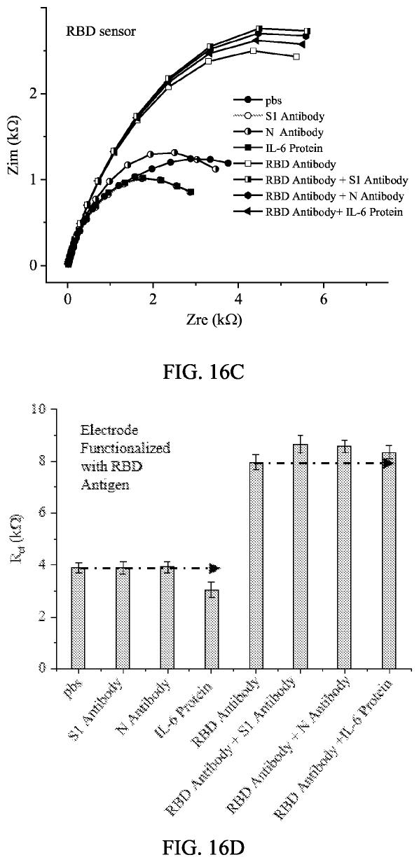

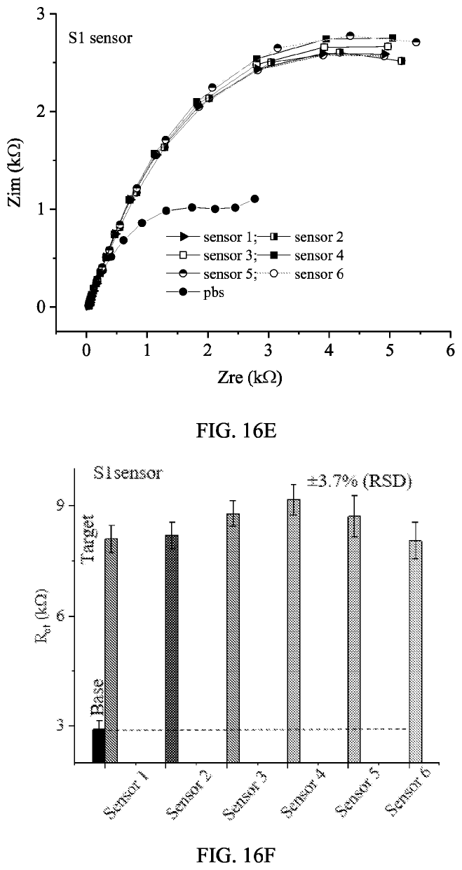

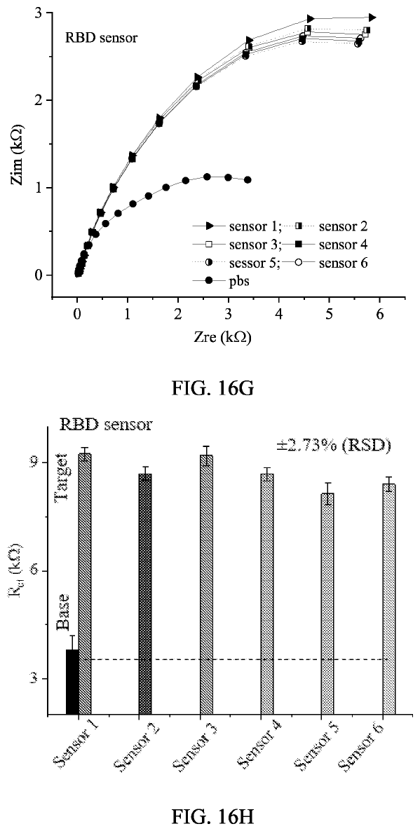

FIGS. 16A and 16B show the Nyquist plots and R_{ct} for cross-reactivity study of the 3DcC sensor designed to test spike S1 antibodies, respectively. The sensor was tested in the presence of RBD antibodies, nucleocapsid (N) antibodies, and cytokines such as interleukin (μL)-6 antigens, without and with the spike S1 antibodies (1 nM). In the absence of spike S1 antibodies, the sensor was found to be insensitive to N antibodies, RBD antibodies, and IL-6 protein molecules as evidenced by its low R_{ct} values and low RSD (±6.9%). The sensor showed a slight deviation (±6.5%) from the initial signal in the presence of RBD antibodies, possibly due to the receptor similarity between the two antibodies. Alternatively, the RBD antibodies might recognize linear epitope while the S1 protein might have a specific confirmation that prevents binding to the RBD antibodies. The sensor was highly sensitive when the spike S1 antibodies were introduced with the above molecules, leading to an increase in the R_{ct} values shown in FIG. 16B. FIGS. 16C and 16D show the cross-reactivity studies of RBD antibodies. Again, we used N antibodies, IL-6 antigens, and spike S1 antibodies in the absence and presence of RBD antigens (1 nM). In this result, the 3DcC device tested with IL-6 antigen showed a deviation of ±11.0% compared to base line with no target. We note that the decrease of R_{ct} value with IL-6 antigen did not affect the sensor response when target antibodies were present in the buffer solution. Without RBD antibodies, the 3D sensor showed an RSD of ±5.81% compared to baseline signal while the RSD was reduced to ±2% in the presence of 1 nM target RBD antibodies. Specific binding sites on the antigen molecules (epitope) reject binding by non-specific molecules but allow binding to a specific antibody (paratope). These results indicated that the sensor showed a good selectivity even within a similar group of proteins.

The sensor reproducibility is shown in FIGS. 16E-16H where twelve 3DcC sensors, 6 for spike S1 antibodies and 6 for RDB antibodies, were evaluated. Nyquist plots were obtained and R_{ct} values were calculated at fixed concentration (1 nm) of spike S1 (FIGS. 16E and 16F) and RBD (FIGS. 16G and 16H) antibodies. For spike S1 and RBD antibodies, the sensors showed a RSD of ±2.7% and ±2.5%, respectively, indicating reasonable reproducibility of the sensor. Note that additive manufacturing (AM) processes are digital (i.e., controlled by CAD programs) and hence are expected to be repeatable. However, AM processes are known to have microstructural inhomogeneities in their final parts. For the 3DcC device, the AJ nanoparticle printing process created repeatable structures (FIGS. 3B and 4A) that provided acceptable repeatability in antibody sensing performance (FIGS. 16E-16H), but led to a microstructure on the pillar surface (high magnification images of FIGS. 3C and 4A) that aided in the surface functionalization process.

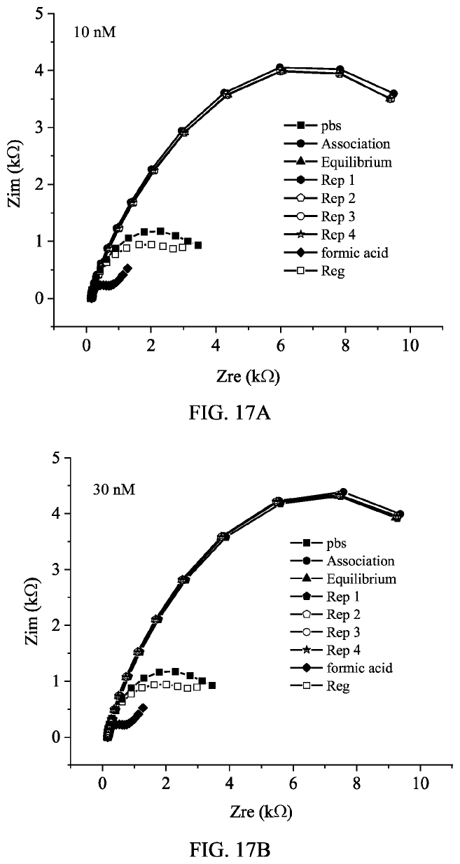

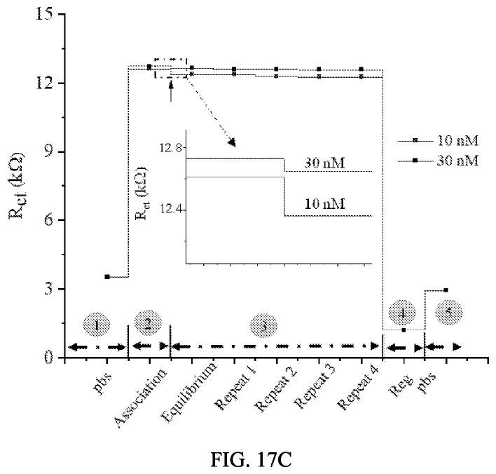

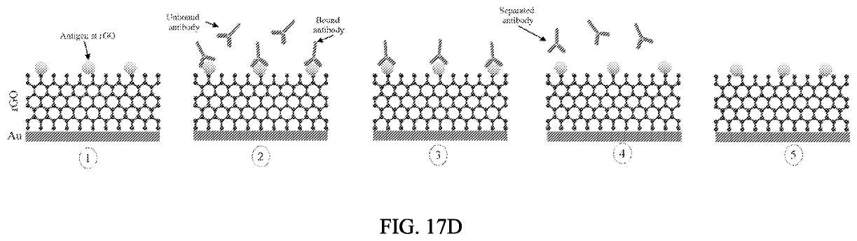

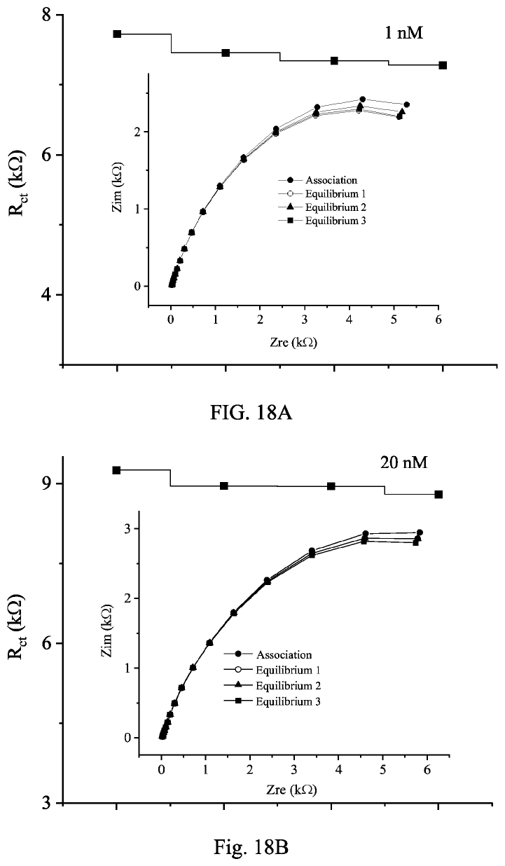

The real-time tracking of binding kinetics of antigens and antibodies at the sensor surface were also investigated. FIGS. 17A-17D show impedance graphs, R_{ct} values, and a schematic explaining the various phases of binding events such as association, equilibrium, and regeneration, respectively. In the association phase, the sensor is exposed to a concentration of target antibodies (spike S1 in this case) where both bound and unbound molecules were present resulting in a slightly higher R_{ct} (FIGS. 17A and 17B). In the equilibrium phase, the sensor was washed with a buffer solution and unbound antibodies were removed from the sensor resulting in a slight reduction in the R_{ct}. However, during regeneration by a low-pH chemistry, elution or desorption of antibodies from the antigens occurred via disruption of ionic and hydrogen bonds in the immunocomplex, reducing the R_{ct}. The signal change from association to equilibrium phase is relatively low. This may be due to the static measurement conditions in the experiments, where the buffer solution is stagnant rather than having an active circulation. The signal is relatively stable over time as shown in the repeatable measurements. A similar result was obtained when two addition sensors for sensing of RBD antibodies were tested (FIGS. 18A and 18B).

### DISCUSSION

The AJ nanoprinted platform (i.e. 3DcC device) developed can detect antibodies for SARS-CoV-2 within seconds. The 3DcC device of FIGS. 3A-3F, which includes the WE with micropillar geometry with surface porosity and a chemistry involving flaky rGO (FIGS. 3A-3G, 4B, and 4C) allowed a high redox current for the electrochemical cell forming the sensor (FIGS. 9A-9G). Further, the 3D geometry allowed a relatively high loading of biorecognition elements (antigens) for the sensor, enabling the detection of the antibodies with LoDs of 2.8 fM and 16.9 fM for spike S1 and RBD antibodies, respectively. FIGS. 16A-16D demonstrate that the natural construction of antigen-antibody immunocomplex makes sensing selective. Another important outcome of this work is the regeneration of the sensor in one minute. By introducing formic acid with a pH of 2.5, the antibodies were eluted from the antigens allowing full recovery of the original sensor signal. The demonstration of 10 repetitions (9× regenerations) of the sensors for each of the antibodies (FIGS. 15C-15F) gives the opportunity for the same sensor to be used multiple times in the field without losing the specificity and sensitivity. We also note that the 3DcC requires only a few μL of fluid for detection of antibodies (e.g., volume obtained by a finger prick with appropriate dilution). Lastly, a convenient smartphone-based user interface (FIG. 3F) will enhance testing accessibility of the device in underdeveloped areas.

The surface features (FIGS. 3C and 4A) of the gold pillars are a result of the AJ nanoprinting process and allows an excellent coverage of the rGO on the electrode surface (e.g., rGO could not be coated on a smooth gold surface). In addition, rGO nanoflakes form a secondary 3D structure (FIG. 4B), allowing high loading of the antigens. The EDC:NHS chemistry used in the work covalently binds the —COOH group of rGO to —NH₂ group of antigens. Therefore, antibodies can be immobilized on the sensor surface and detect pathogens directly via their antigens. The AJ nanoprinting is a digital manufacturing platform where the sensor design is controlled by CAD programs. The fabrication is also rapid—the 10×10 gold pillar array (FIG. 2b) takes 35 minutes to print per printhead. With the availability of AJ printers with four printheads working in tandem, the effective printing time can be reduced to 7-8 minutes per sensor followed by a batch sintering process. Such digital manufacture allows fabrication in two simple steps without the need for any cleanroom processes, lowering resources/cost per sensor that can improve accessibility of testing. In addition, 3D printing enables the sensor design to be changed via simple changes to CAD programs.

The 3DcC device has the ability to detect SARS-CoV-2 antibodies non-destructively within seconds at low analytical sensitivities with label-free probing of antibodies and an ability for regeneration within one minute. A signal was obtained in all the 3DcC devices used every time an antibody solution was introduced (FIGS. 11A-11D, 13A-13D, 15A-15D, 16A-16H, and 17A-17D). A LoD at femtomolar concentrations for antibodies may enable an early detection of the disease.

### Example 2

A gold micropillar array for a working electrode was prepared using AJ printing, as described above in Example 1. The gold micropillars were functionalized with rGO as described above in Example 1. The Au micropillar-rGO surface was further functionalized using SARS-CoV-2 antibodies to the Spike S1 antigens. This was achieved by using a coupling reagent consisting of a mixture of EDC (0.2 M) and NHS (0.05 M) in a ratio of 1:1 by volume. A 20 μL solution of the EDC:NHS mixture was spread over the rGO-Au surface to activate the —COOH groups of the rGO sheets. The electrode on the glass substrate was kept in a humid chamber (at approximately 100% of humidity) for four hours and washed with PBS solution. Next, a 20 μL of SARS-CoV-2 spike S1 antibody solution (5 μg/mL) was spread on the surface of the rGO-Au array electrode via drop-casting using a pipette (10-50 μL) and kept for 4 hours in a humid chamber and then washed with PBS. The activation achieved by EDC:NHS chemistry enabled the primary amine (—NH₂) groups of the antibody of SARS-CoV-2 spike to form C—N bonds with the —COOH groups of rGO sheets via an amidation reaction.

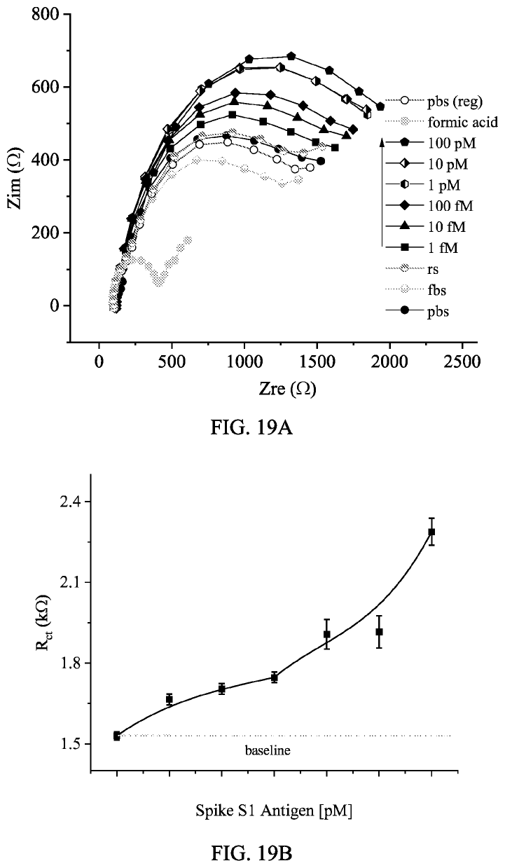

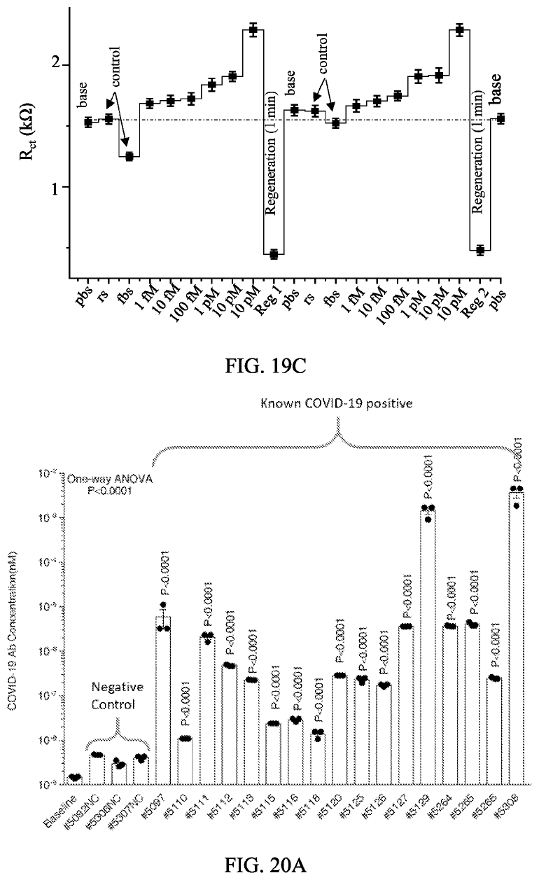

SAR-CoV-2 spike S1 antigens were detected using the methods as described above in Example 1. FIGS. 19A-19B shows the impedimetric sensing plots and R_{ct} for the 3DcC sensor when the electrodes are exposed to PBS and spike S1 antigen. The spike S1 antigens were introduced successively at concentrations of 1 fM, 10 fM, 100 fM, 1 pM, and 100 pM. In the first set of measurements, a testing baseline was set by allowing only the PBS solution with no target antigens which showed a charge transferred-limited process. The sensor is also tested with rs and fbs solution to check the sensor cross-reactivity. The 3DcC device was tested with low concentrations of spike S1 antigen at 1 fM and 10 fM. With the 1 fM concentration, a change of impedance signal was observed compared to the sensor baseline. At 100 pM concentration of antigens, however, the sensor showed a significant increase in the impedance (FIG. 19A). As the antigen concentration was increased, the R_{ct} increased further as reflected in the Nyquist plots (FIG. 19B).

Upon introduction of a formic acid solution for 60 seconds into the sensor, the R_{ct} values dropped (FIG. 19C). In addition to eluting the antigens, the low R_{ct} is caused by high electron transfer rates in acidic media. The signal from formic acid are after the 1^{st} and 2^{nd} regeneration. After the 60 second incubation by formic acid solution, the sensor was washed with a PBS solution, rabbit serum, and fetal bovine serum.

### Example 3

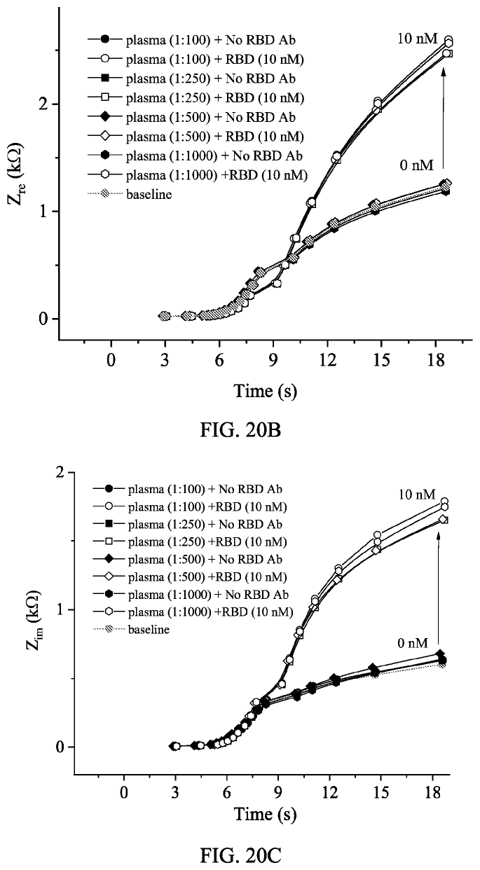

The gold micropillar array prepared in Example 1 was used to detect SARS-CoV-2 RBD antibodies in human serum samples. The human serum samples tested were collected from individuals that tested positive (17 samples) and negative (3 samples) for COVID-19. Antibodies were detected in the COVID-positive human serum samples as shown in FIG. 20A. FIG. 20B shows the real impedance signal of the sensor as a function of time (in seconds) for the detection of spiked plasma where 10 nM RBD antibodies were added into a set of diluted human plasma. FIG. 20C shows the impedance signal of the sensor as a function of time (in seconds) for the detection of spiked plasma where a 10 nM RBD antibodies were added into a set of diluted human plasma.

Having described this invention above, it will be understood to those of ordinary skill in the art that the same can be performed within a wide and equivalent range of conditions, formulations and other parameters without affecting the scope of the invention or any embodiment thereof.

1. A method of preparing a functionalized electrode, comprising:
depositing a conductive material onto the surface of a substrate by droplet-based printing, such as aerosol jet printing, of particles comprising an electrically-conductive material, and
functionalizing the surface of the conductive material with a binding reagent that binds to an analyte.

2. The method of claim 1, wherein the conductive material is deposited as a plurality of protuberances onto the surface of the substrate, and optionally wherein the protuberances have a diameter of not greater than 10 millimeters, and the area of the substrate comprising the protuberances is less than or equal to 200 square millimeters (mm²) and comprises at least one protuberance per mm².

3. (canceled)

4. (canceled)

5. The method of claim 1, wherein the conductive material is deposited as an ink comprising nanoparticles or microparticles that are optionally deposited by aerosol jet printing.

6. (canceled)

7. The method of claim 5, wherein the particles are nanoparticles having a diameter of at least 4 nanometers to not greater than 1 micron.

8. (canceled)

9. (canceled)

10. The method of claim 1, wherein the droplets comprise a solvent, and substrate is maintained at a temperature of 50° C. or greater during the deposition of the protuberances to evaporate the solvent.

11. The method of claim 1, wherein the electrically-conductive material of the particles comprises gold, silver, platinum, nickel, rhodium, zinc, an alloy of any of the preceding, carbon, a conductive polymer, graphene, such as graphene oxide, molybdenum disulfide (MoS₂), MXenes, such as titanium carbide, or any combination thereof.

12. (canceled)

13. (canceled)

14. The method of claim 2, wherein the protuberances are individual pillars, optionally having a height ranging from 1 micron to 1,000 microns and a diameter ranging from 0.1 microns to 500 microns.

15. (canceled)

16. The method of claim 2, wherein the protuberances form an open cell lattice.

17. (canceled)

18. The method of claim 1, further comprising sintering the deposited conductive material optionally at a temperature above 100° C. for at least 10 minutes.

19. (canceled)

20. The method of claim 1, further comprising coating the deposited conductive material with an electrically active material, such as graphite, hard carbon, synthetic graphite, carbon black, graphene, such as graphene oxide, carbon nanotubes, gold, molybdenum disulfide (MoS₂), MXenes, such as titanium carbide, or any combination thereof.

21. (canceled)

22. (canceled)

23. The method of claim 1, further comprising coating the deposited conductive material with a linking molecule comprising a first portion, a second portion, and a linking portion,
wherein the first portion of the linking molecule comprises a functional group for attachment of the linking molecule to the surface of the protuberance, the second portion comprises a functional group for attachment of the linking molecule to the binding reagent, and the linking portion of the molecule extends between the first portion and the second portion, and wherein the linking molecule is optionally (3-aminopropyl)triethoxysilane (APTES), L-Cysteine, thioglycolic acid, poly(ethylene glycol), N-hydroxysuccinimide esters, 11-mercaptoundecanoic acid, 12-mercaptodeodecanoic acid, or any combination thereof.

24. (canceled)

25. The method of claim 23, further comprising reacting the second portion of the linking molecule with the binding reagent, to link the binding reagent to the deposited conductive material, wherein the binding reagent optionally comprises: a protein, such as a lectin; an antibody or an antibody fragment; an epitope-containing polypeptide, an antigen; an aptamer, an affimer, a nucleic acid or any combination of the preceding, such as a protein of a coronavirus, such as SARS-CoV-2 ebola virus, human immunodeficiency virus (HIV), influenza virus, herpes virus, zika virus, Escherichia coli, or Mycobacterium tuberculosis.

26-28. (canceled)

29. An electrode, comprising:
a substrate comprising a droplet-based printed, and optionally sintered, conductive material, and
a coating comprising a binding reagent covalently bonded to the surface of the conductive material that binds to an analyte, wherein the deposited conductive material optionally comprises gold, silver, platinum, nickel, rhodium, zinc, alloys of any of the preceding, carbon, a conductive polymer, graphene, such as graphene oxide, molybdenum disulfide (MoS₂), MXenes, such as titanium carbide, or any combination thereof.

30. (canceled)

31. The electrode of claim 29, wherein the conductive material is deposited as a plurality of protuberances onto the surface of the substrate, optionally wherein the area of the substrate comprising the protuberances is less than or equal to 200 square millimeters (mm²) and comprises at least 1 protuberance per mm².

32. (canceled)

33. (canceled)

34. The electrode of claim 31, wherein the protuberances are individual pillars.

35. The electrode of claim 34, wherein the individual pillars have a height ranging from 1 micron to 1,000 microns and a diameter ranging from 0.1 microns to 500 microns.

36. (canceled)

37. (canceled)

38. The electrode of claim 29, wherein the binding reagent comprises: a protein, such as a lectin; an antibody, an antibody fragment, or an engineered antibody, e.g., an scFv; an epitope-containing polypeptide, an antigen; an aptamer, a nucleic acid, or any combination of any of the preceding, such as an antigen or epitope of a protein of a virus, a bacteria, a fungus, or a parasite, such as a protein of a coronavirus, such as SARS-CoV-2, ebola virus, human immunodeficiency virus (HIV), influenza virus, herpes virus, zika virus, Escherichia coli, or Mycobacterium tuberculosis.

39. (canceled)

40. (canceled)

41. A microfluidic test device comprising:
one or more sensing electrodes in a chamber or channel configured to receive a liquid test sample,
wherein the sensing electrode comprises a substrate and an electrode array comprising a working electrode, a counter electrode, and, optionally, a reference electrode on the substrate, and the working electrode comprises
the electrode of claim 29.

42-52. (canceled)

53. A method of sensing an analyte, the method comprising:
contacting a fluid comprising the analyte with the electrode of claim 29.

54-60. (canceled)
